(19) United States
(12) Patent Application Publication
(10) Pub. No.: US 2018/0325610 A1
(43) Pub. Date: Nov. 15, 2018

(54) METHODS FOR INDICATING AND CONFIRMING A POINT OF INTEREST USING SURGICAL NAVIGATION SYSTEMS

(71) Applicant: GLOBUS MEDICAL, INC., AUDUBON, PA (US)

(72) Inventors: Hayden Cameron, Philadelphia, PA (US); Andrew Berkowitz, Philadelphia, PA (US); Neil R. Crawford, Chandler, AZ (US)

(21) Appl. No.: 16/035,779

(22) Filed: Jul. 16, 2018

Related U.S. Application Data
(63) Continuation-in-part of application No. 15/609,334, filed on May 31, 2017.
Application No. 15/609,334 is a continuation-in-part of application No. 15/157,444, filed on May 18, 2016.
Application No. 15/157,444 is a continuation-in-part of application No. 15/095,883, filed on Apr. 11, 2016.
Application No. 15/095,883 is a continuation-in-part of application No. 14/062,707, filed on Oct. 24, 2013.
Application No. 14/062,707 is a continuation-in-part of application No. 13/924,505, filed on Jun. 21, 2013, now Pat. No. 9,782,229.
(60) Provisional application No. 61/662,702, filed on Jun. 21, 2012.
(60) Provisional application No. 61/800,527, filed on Mar. 15, 2013.

(51) Int. Cl. A61B 34/30; A61B 5/06; A61B 90/00; A61B 90/11; G06T 7/73

(52) U.S. Cl. CPC A61B 34/30; A61B 5/064; A61B 90/98; A61B 90/11; G06T 7/73; A61B 90/37

(57) ABSTRACT

Methods may be provided to operate an image-guided surgical system using imaging information for a 3-dimensional anatomical volume. A trigger can be detected based on information received from a tracking system regarding a probe. A pose of the probe can be detected based on the information received from the tracking system. A location in the 3-dimensional anatomical volume can be identified based on the pose of the probe and based on the imaging information. The location can be stored in memory based on detecting the trigger and detecting the pose of the probe.

### CROSS REFERENCE TO RELATED APPLICATIONS

This application is a continuation-in-part of U.S. patent application Ser. No. 15/609,334 which is a continuation-in-part of U.S. patent application Ser. No. 15/157,444, filed May 18, 2016, which is a continuation-in-part of U.S. patent application Ser. No. 15/095,883, filed Apr. 11, 2016, which is a continuation-in-part of U.S. patent application Ser. No. 14/062,707, filed on Oct. 24, 2013, which is a continuation-in-part application of U.S. patent application Ser. No. 13/924,505, filed on Jun. 21, 2013, which claims priority to provisional application No. 61/662,702 filed on Jun. 21, 2012 and claims priority to provisional application No. 61/800,527 filed on Mar. 15, 2013, all of which are incorporated by reference herein in their entireties for all purposes.

### FIELD

The present disclosure relates to medical devices, and more particularly, surgical navigation systems and related methods and devices.

### BACKGROUND

Medical imaging systems may be used to generate images of a three-dimensional (3D) volume of a body. Position recognition systems can be used to determine the position of and track a particular object in 3D space. In robot assisted surgeries, for example, certain objects, such as surgical instruments, may need to be tracked with a high degree of precision as the instrument is being positioned and moved by a robot or by a physician.

Infrared signal based position recognition systems may use passive and/or active sensors or markers to track the objects. With respect to passive sensors or markers, objects to be tracked may include passive sensors, such as reflective spherical balls, which are positioned at strategic locations on the object to be tracked. Infrared transmitters transmit a signal, and the reflective spherical balls reflect the signal to aid in determining the position of the object in 3D. With respect to active sensors or markers, the objects to be tracked include active infrared transmitters, such as light emitting diodes (LEDs), and thus generate their own infrared signals for 3D detection. With either active or passive tracking sensors, the system then geometrically resolves the 3D position of the active and/or passive sensors based on information from or with respect to one or more of the infrared cameras, digital signals, known locations of the active or passive sensors, distance, the time it took to receive the responsive signals, other known variables, or a combination thereof.

These surgical systems can therefore utilize position feedback to precisely guide movement of robotic arms and tools relative to a patient's surgical site. However, these systems may include limited ability to virtually plan and prepare for the placement of some tools and implants. In some examples, the 3D position and pose or orientation of a probe can be tracked by a tracking system and a virtual version displayed on the monitor with respect to an image of the patient. But, it can be desirable to indicate and store a specific virtual location on a patient that may be subcutaneous and inaccessible without making an incision or burr hole.

### SUMMARY

According to some embodiments of inventive concepts, methods may be provided to operate an image-guided surgical system using anatomical imaging information for a 3-dimensional (3D) anatomical volume. A trigger can be detected based on information received from a tracking system regarding a probe. A pose of the probe can be detected based on the information received from the tracking system. A location in the 3D anatomical volume can be identified based on the pose of the probe and based on the imaging information. The location can be stored in memory based on detecting the trigger and detecting the pose of the probe.

According to other embodiments of inventive concepts, another method may be provided to operate an image-guided surgical system using anatomical imaging information for a 3D anatomical volume. A first distance from an end of a probe can be defined. A first pose of the probe can be detected based on information received from a tracking system. A first location in the 3D anatomical volume can be identified based on the first pose of the probe, the imaging information, and the first distance from the end of the probe. A trigger can be detected based on the information received from the tracking system regarding the probe after identifying the first location. A second distance from the end of the probe can be defined in response to detecting the trigger. A second pose of the probe can be detected based on the information received from the tracking system. A second location in the 3D anatomical volume can be identified based on the second pose of the probe, the imaging information, and the second distance from the end of the probe.

According to still other embodiments of inventive concepts, an image-guided surgical system using 3D anatomical imaging information for a 3D anatomical volume can include a processor and a memory coupled with the processor. The memory can include instructions stored therein. The instructions can be executed by the processor to cause the processor to detect a trigger based on information received from a tracking system regarding a probe. The instructions can further cause the processor to detect a pose of the probe based on the information received from the tracking system. The instructions can further cause the processor to identify a location in the 3D anatomical volume based on the pose of the probe and based on the imaging information. The instructions can further cause the processor to store the location in memory based on detecting the trigger and detecting the pose of the probe.

According to still other embodiments of inventive concepts, an image-guided surgical system using 3D anatomical imaging information for a 3D anatomical volume can include a processor and a memory coupled with the processor. The memory can include instructions stored therein. The instructions can be executed by the processor to cause the processor to define a first distance from an end of a probe. The instructions can further cause the processor to detect a first pose of the probe based on information received from the tracking system. The instructions can further cause the processor to identify a first location in the 3D anatomical volume based on the first pose of the probe, the imaging information, and the first distance from an end of the probe. The instructions can further cause the processor to detect a trigger based on information received from the tracking system regarding the probe after identifying the first location. The instructions can further cause the processor to define a second distance from the end of the probe responsive to detecting the trigger. The instructions can further cause the processor to detect a second pose of the probe based on information received from the tracking system. The instructions can further cause the processor to identify a second location in the 3D anatomical volume based on the second pose of the probe, the imaging information, and the second distance from the end of the probe.

Other methods and related image-guided surgical systems, and corresponding methods and computer program products according to embodiments of the inventive subject matter will be or become apparent to one with skill in the art upon review of the following drawings and detailed description. It is intended that all such image-guided surgical systems, and corresponding methods and computer program products be included within this description, be within the scope of the present inventive subject matter, and be protected by the accompanying claims. Moreover, it is intended that all embodiments disclosed herein can be implemented separately or combined in any way and/or combination.

### DESCRIPTION OF THE DRAWINGS

The accompanying drawings, which are included to provide a further understanding of the disclosure and constitute a part of this application, illustrate certain non-limiting embodiments of inventive concepts. In the drawings:

FIG. 1 is an overhead view of a potential arrangement for locations of the robotic system, patient, doctor, and other medical personnel during a medical procedure;

FIG. 2 illustrates the robotic system including positioning of the medical robot and the camera relative to the patient according to some embodiments;

FIG. 3 illustrates a medical robotic system in accordance with some embodiments;

FIG. 4 illustrates a portion of a medical robot in accordance with some embodiments;

FIG. 5 illustrates a block diagram of a medical robot in accordance with some embodiments;

FIG. 6 illustrates a medical robot in accordance with some embodiments;

FIGS. 7A-7C illustrate an end-effector in accordance with some embodiments;

FIG. 8 illustrates a medical instrument and the end-effector, before and after, inserting the medical instrument into the guide tube of the end-effector according to some embodiments;

FIGS. 9A-9C illustrate portions of an end-effector and robot arm in accordance with some embodiments;

FIG. 10 illustrates a dynamic reference array, an imaging array, and other components in accordance with some embodiments;

FIG. 11 illustrates a method of registration in accordance with some embodiments;

FIGS. 12A-12B illustrate embodiments of imaging devices according to some embodiments;

FIG. 13A illustrates a portion of a robot including the robot arm and an end-effector in accordance with some embodiments;

FIG. 13B is a close-up view of the end-effector, with a plurality of tracking markers rigidly affixed thereon, shown in FIG. 13A;

FIG. 13C is a tool or instrument with a plurality of tracking markers rigidly affixed thereon according to some embodiments;

FIG. 14A is an alternative version of an end-effector with moveable tracking markers in a first configuration;

FIG. 14B is the end-effector shown in FIG. 14A with the moveable tracking markers in a second configuration;

FIG. 14C shows the template of tracking markers in the first configuration from FIG. 14A;

FIG. 14D shows the template of tracking markers in the second configuration from FIG. 14B;

FIG. 15A shows an alternative version of the end-effector having only a single tracking marker affixed thereto;

FIG. 15B shows the end-effector of FIG. 15A with an instrument disposed through the guide tube;

FIG. 15C shows the end-effector of FIG. 15A with the instrument in two different positions, and the resulting logic to determine if the instrument is positioned within the guide tube or outside of the guide tube;

FIG. 15D shows the end-effector of FIG. 15A with the instrument in the guide tube at two different frames and its relative distance to the single tracking marker on the guide tube;

FIG. 15E shows the end-effector of FIG. 15A relative to a coordinate system;

FIG. 16 is a block diagram of a method for navigating and moving the end-effector of the robot to a desired target trajectory;

FIGS. 17A-17B depict an instrument for inserting an expandable implant having fixed and moveable tracking markers in contracted and expanded positions, respectively;

FIGS. 18A-18B depict an instrument for inserting an articulating implant having fixed and moveable tracking markers in insertion and angled positions, respectively;

FIGS. 19A depicts an embodiment of a robot with interchangeable or alternative end-effectors;

FIG. 19B depicts an embodiment of a robot with an instrument style end-effector coupled thereto;

FIG. 20 is a block diagram illustrating a controller according to some embodiments;

FIG. 21 illustrates an image-guided surgical system detecting a probe and determining a location of a virtual crosshair according to some embodiments;

FIGS. 22A-B depict examples of a telescoping probe for triggering an image system to store a location or manipulate a 3D image according to some embodiments;

FIGS. 23-24 are flow charts illustrating examples operations of an image-guided surgical system according to some embodiments; and

FIGS. 25, 26A-B, and 27A-B depict examples of movements of a probe that may be detected by an image-guided surgical system as triggers according to some embodiments.

### DETAILED DESCRIPTION

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

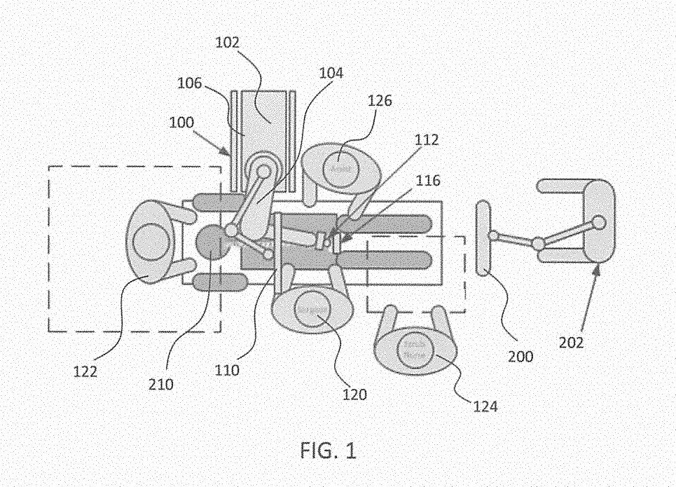

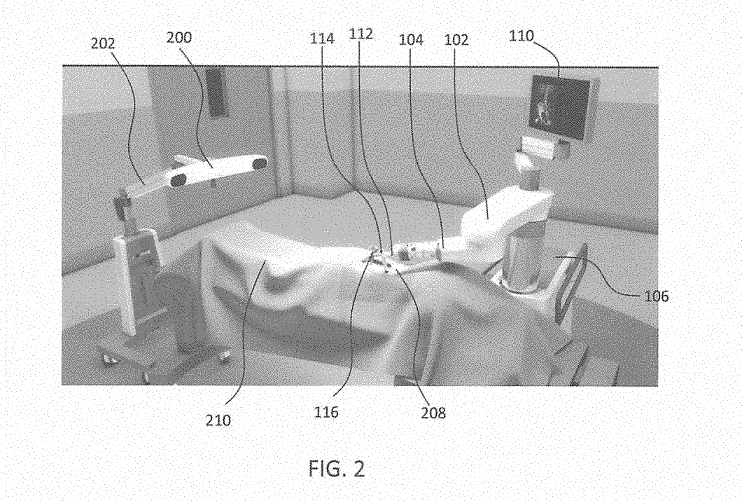

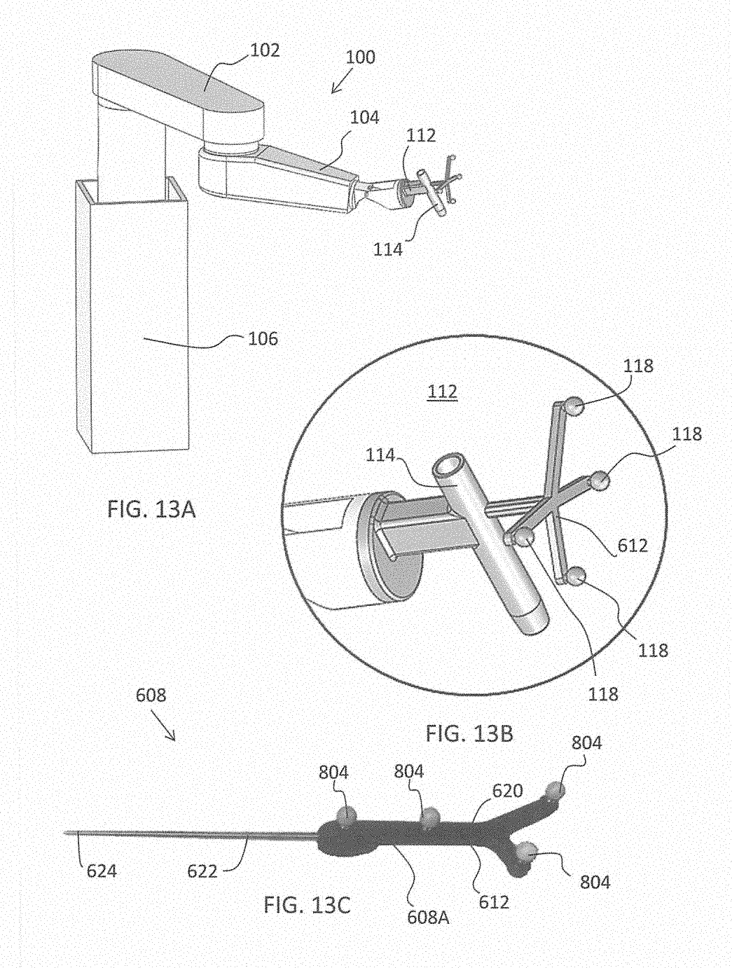

Turning now to the drawings, FIGS. 1 and 2 illustrate a medical robot system 100 in accordance with some embodiments. Medical robot system 100 may include, for example, a medical robot 102, one or more robot arms 104, a base 106, a display 110, an end-effector 112, for example, including a guide tube 114, and one or more tracking markers 118. The medical robot system 100 may include a patient tracking device 116 also including one or more tracking markers 118, which is adapted to be secured directly to the patient 210 (e.g., to a bone of the patient 210). The medical robot system 100 may also use a camera(s) 200, for example, positioned on a camera stand 202. The camera stand 202 can have any suitable configuration to move, orient, and support the camera 200 in a desired position. The camera 200 may include any suitable camera or cameras, such as one or more infrared cameras (e.g., bifocal or stereophotogrammetric cameras), able to identify, for example, active and passive tracking markers 118 (shown as part of patient tracking device 116 in FIG. 2 and shown by enlarged view in FIGS. 13A-13B) in a given measurement volume viewable from the perspective of the camera 200. The camera 200 may scan the given measurement volume and detect the light that comes from the markers 118 in order to identify and determine the position of the markers 118 in three-dimensions. For example, active markers 118 may include infrared-emitting markers that are activated by an electrical signal (e.g., infrared light emitting diodes (LEDs)), and/or passive markers 118 may include retro-reflective markers that reflect infrared light (e.g., they reflect incoming IR radiation into the direction of the incoming light), for example, emitted by illuminators on the camera 200 or other suitable device.

FIGS. 1 and 2 illustrate a potential configuration for the placement of the medical robot system 100 in an operating room environment. For example, the robot 102 may be positioned near or next to patient 210. Although depicted near the head of the patient 210, it will be appreciated that the robot 102 can be positioned at any suitable location near the patient 210 depending on the area of the patient 210 undergoing the operation. The camera 200 may be separated from the robot system 100 and positioned at the foot of patient 210. This location allows the camera 200 to have a direct visual line of sight to the medical field 208. Again, it is contemplated that the camera 200 may be located at any suitable position having line of sight to the medical field 208. In the configuration shown, the doctor 120 may be positioned across from the robot 102, but is still able to manipulate the end-effector 112 and the display 110. A medical assistant 126 may be positioned across from the doctor 120 again with access to both the end-effector 112 and the display 110. If desired, the locations of the doctor 120 and the assistant 126 may be reversed. The traditional areas for the anesthesiologist 122 and the nurse or scrub tech 124 may remain unimpeded by the locations of the robot 102 and camera 200.

With respect to the other components of the robot 102, the display 110 can be attached to the medical robot 102 and in some embodiments, display 110 can be detached from medical robot 102, either within a surgical room (or other medical facility) with the medical robot 102, or in a remote location. End-effector 112 may be coupled to the robot arm 104 and controlled by at least one motor. In some embodiments, end-effector 112 can comprise a guide tube 114, which is able to receive and orient a medical instrument 608 (described further herein) used on the patient 210. As used herein, the term “end-effector” is used interchangeably with the terms “end-effectuator” and “effectuator element.” Although generally shown with a guide tube 114, it will be appreciated that the end-effector 112 may be replaced with any suitable instrumentation suitable for use in surgery. In some embodiments, end-effector 112 can comprise any known structure for effecting the movement of the medical instrument 608 in a desired manner.

The medical robot 102 is able to control the translation and orientation of the end-effector 112. The robot 102 is able to move end-effector 112 along x-, y-, and z-axes, for example. The end-effector 112 can be configured for selective rotation about one or more of the x-, y-, and z-axis, and a Z Frame axis (such that one or more of the Euler Angles (e.g., roll, pitch, and/or yaw) associated with end-effector 112 can be selectively controlled). In some embodiments, selective control of the translation and orientation of end-effector 112 can permit performance of medical procedures with significantly improved accuracy compared to conventional robots that use, for example, a six degree of freedom robot arm comprising only rotational axes. For example, the medical robot system 100 may be used to provide medical imaging and/or to operate on patient 210, and robot arm 104 can be positioned above the body of patient 210, with end-effector 112 selectively angled relative to the z-axis toward the body of patient 210.

In some embodiments, the position of the medical instrument 608 can be dynamically updated so that medical robot 102 can be aware of the location of the medical instrument 608 at all times during the procedure. Consequently, in some embodiments, medical robot 102 can move the medical instrument 608 to the desired position quickly without any further assistance from a physician (unless the physician so desires). In some further embodiments, medical robot 102 can be configured to correct the path of the medical instrument 608 if the medical instrument 608 strays from the selected, preplanned trajectory. In some embodiments, medical robot 102 can be configured to permit stoppage, modification, and/or manual control of the movement of end-effector 112 and/or the medical instrument 608. Thus, in use, in some embodiments, a physician or other user can operate the system 100, and has the option to stop, modify, or manually control the autonomous movement of end-effector 112 and/or the medical instrument 608. Further details of medical robot system 100 including the control and movement of a medical instrument 608 by medical robot 102 can be found in co-pending U.S. patent application Ser. No. 13/924,505, which is incorporated herein by reference in its entirety.

The robotic medical system 100 can comprise one or more tracking markers 118 configured to track the movement of robot arm 104, end-effector 112, patient 210, and/or the medical instrument 608 in three dimensions. In some embodiments, a plurality of tracking markers 118 can be mounted (or otherwise secured) thereon to an outer surface of the robot 102, such as, for example and without limitation, on base 106 of robot 102, on robot arm 104, and/or on the end-effector 112. In some embodiments, at least one tracking marker 118 of the plurality of tracking markers 118 can be mounted or otherwise secured to the end-effector 112. One or more tracking markers 118 can further be mounted (or otherwise secured) to the patient 210. In some embodiments, the plurality of tracking markers 118 can be positioned on the patient 210 spaced apart from the medical field 208 to reduce the likelihood of being obscured by the doctor, medical tools, or other parts of the robot 102. Further, one or more tracking markers 118 can be further mounted (or otherwise secured) to the medical tools 608 (e.g., an ultrasound transducer, a screw driver, dilator, implant inserter, or the like). Thus, the tracking markers 118 enable each of the marked objects (e.g., the end-effector 112, the patient 210, and the medical tools 608) to be tracked by the robot 102. In some embodiments, system 100 can use tracking information collected from each of the marked objects to calculate the orientation and location, for example, of the end-effector 112, the medical instrument 608 (e.g., positioned in the tube 114 of the end-effector 112), and the relative position of the patient 210.

The markers 118 may include radiopaque or optical markers. The markers 118 may be suitably shaped include spherical, spheroid, cylindrical, cube, cuboid, or the like. In some embodiments, one or more of markers 118 may be optical markers. In some embodiments, the positioning of one or more tracking markers 118 on end-effector 112 can increase/maximize the accuracy of the positional measurements by serving to check or verify the position of end-effector 112. Further details of medical robot system 100 including the control, movement and tracking of medical robot 102 and of a medical instrument 608 can be found in U.S. patent publication No. 2016/0242849, which is incorporated herein by reference in its entirety.

Some embodiments include one or more markers 118 coupled to the medical instrument 608. In some embodiments, these markers 118, for example, coupled to the patient 210 and medical instruments 608, as well as markers 118 coupled to the end-effector 112 of the robot 102 can comprise conventional infrared light-emitting diodes (LEDs) or an Optotrak® diode capable of being tracked using a commercially available infrared optical tracking system such as Optotrak®. Optotrak® is a registered trademark of Northern Digital Inc., Waterloo, Ontario, Canada. In other embodiments, markers 118 can comprise conventional reflective spheres capable of being tracked using a commercially available optical tracking system such as Polaris Spectra. Polaris Spectra is also a registered trademark of Northern Digital, Inc. In some embodiments, the markers 118 coupled to the end-effector 112 are active markers which comprise infrared light-emitting diodes which may be turned on and off, and the markers 118 coupled to the patient 210 and the medical instruments 608 comprise passive reflective spheres.

In some embodiments, light emitted from and/or reflected by markers 118 can be detected by camera 200 and can be used to monitor the location and movement of the marked objects. In alternative embodiments, markers 118 can comprise a radio-frequency and/or electromagnetic reflector or transceiver and the camera 200 can include or be replaced by a radio-frequency and/or electromagnetic transceiver.

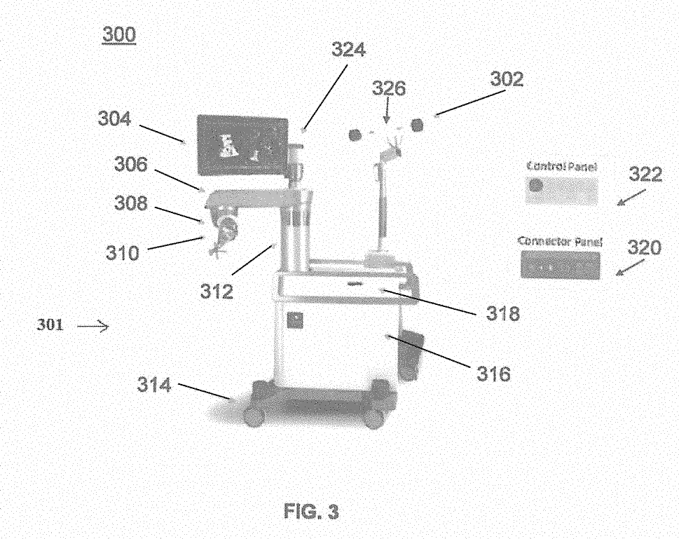

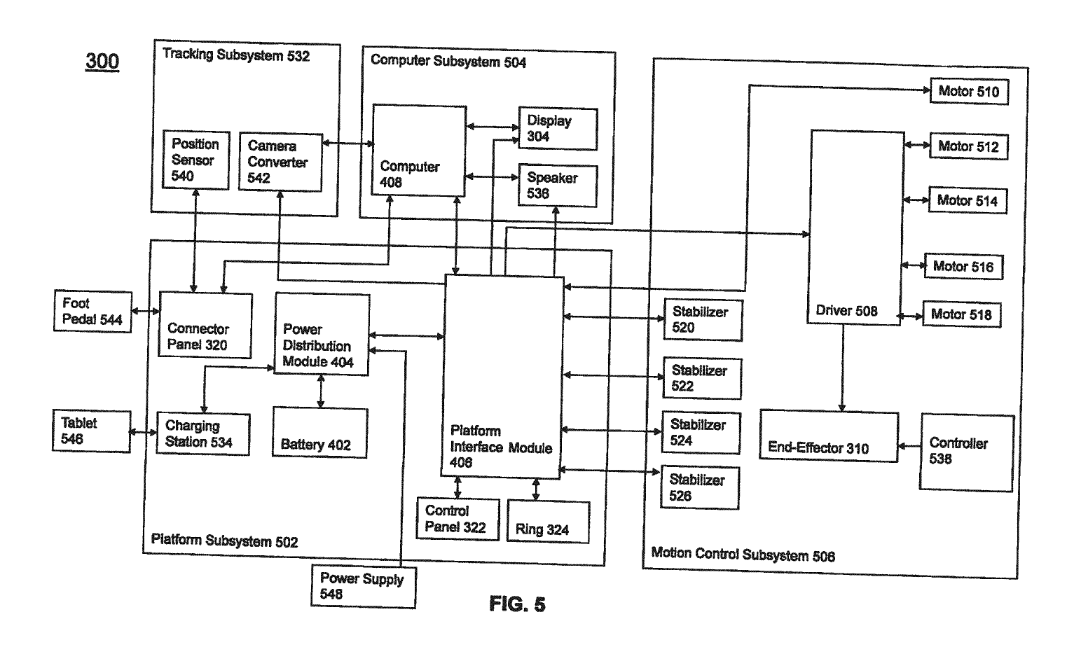

Similar to medical robot system 100, FIG. 3 illustrates a medical robot system 300 and camera stand 302, in a docked configuration, consistent with some embodiments of the present disclosure. Medical robot system 300 may comprise a robot 301 including a display 304, upper arm 306, lower arm 308, end-effector 310, vertical column 312, casters 314, cabinet 316, tablet drawer 318, connector panel 320, control panel 322, and ring of information 324. Camera stand 302 may comprise camera 326. These components are described in greater with respect to FIG. 5. FIG. 3 illustrates the medical robot system 300 in a docked configuration where the camera stand 302 is nested with the robot 301, for example, when not in use. It will be appreciated by those skilled in the art that the camera 326 and robot 301 may be separated from one another and positioned at any appropriate location during the medical procedure, for example, as shown in FIGS. 1 and 2.

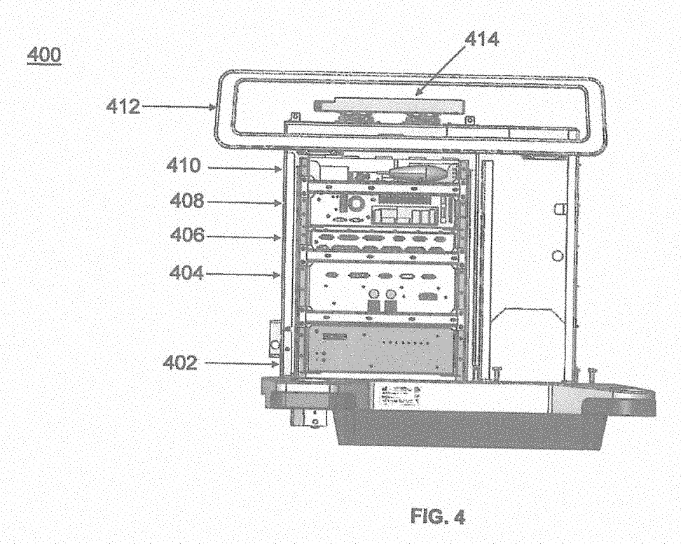

FIG. 4 illustrates a base 400 consistent with some embodiments of the present disclosure. Base 400 may be a portion of medical robot system 300 and comprise cabinet 316. Cabinet 316 may house certain components of medical robot system 300 including but not limited to a battery 402, a power distribution module 404, a platform interface board module 406, a computer 408, a handle 412, and a tablet drawer 414. The connections and relationship between these components is described in greater detail with respect to FIG. 5.

FIG. 5 illustrates a block diagram of certain components of some embodiments of medical robot system 300. Medical robot system 300 may comprise platform subsystem 502, computer subsystem 504, motion control subsystem 506, and tracking subsystem 532. Platform subsystem 502 may further comprise battery 402, power distribution module 404, platform interface board module 406, and tablet charging station 534. Computer subsystem 504 may further comprise computer 408, display 304, and speaker 536. Motion control subsystem 506 may further comprise driver circuit 508, motors 510, 512, 514, 516, 518, stabilizers 520, 522, 524, 526, end-effector 310, and controller 538. Tracking subsystem 532 may further comprise position sensor 540 and camera converter 542. System 300 may also comprise a foot pedal 544 and tablet 546.

Input power is supplied to system 300 via a power source 548 which may be provided to power distribution module 404. Power distribution module 404 receives input power and is configured to generate different power supply voltages that are provided to other modules, components, and subsystems of system 300. Power distribution module 404 may be configured to provide different voltage supplies to platform interface module 406, which may be provided to other components such as computer 408, display 304, speaker 536, driver 508 to, for example, power motors 512, 514, 516, 518 and end-effector 310, motor 510, ring 324, camera converter 542, and other components for system 300 for example, fans for cooling the electrical components within cabinet 316.

Power distribution module 404 may also provide power to other components such as tablet charging station 534 that may be located within tablet drawer 318. Tablet charging station 534 may be in wireless or wired communication with tablet 546 for charging table 546. Tablet 546 may be used by a doctor consistent with the present disclosure and described herein.

Power distribution module 404 may also be connected to battery 402, which serves as temporary power source in the event that power distribution module 404 does not receive power from input power 548. At other times, power distribution module 404 may serve to charge battery 402 if necessary.

Other components of platform subsystem 502 may also include connector panel 320, control panel 322, and ring 324. Connector panel 320 may serve to connect different devices and components to system 300 and/or associated components and modules. Connector panel 320 may contain one or more ports that receive lines or connections from different components. For example, connector panel 320 may have a ground terminal port that may ground system 300 to other equipment, a port to connect foot pedal 544 to system 300, a port to connect to tracking subsystem 532, which may comprise position sensor 540, camera converter 542, and cameras 326 associated with camera stand 302. Connector panel 320 may also include other ports to allow USB, Ethernet, HDMI communications to other components, such as computer 408.

Control panel 322 may provide various buttons or indicators that control operation of system 300 and/or provide information regarding system 300. For example, control panel 322 may include buttons to power on or off system 300, lift or lower vertical column 312, and lift or lower stabilizers 520-526 that may be designed to engage casters 314 to lock system 300 from physically moving. Other buttons may stop system 300 in the event of an emergency, which may remove all motor power and apply mechanical brakes to stop all motion from occurring. Control panel 322 may also have indicators notifying the user of certain system conditions such as a line power indicator or status of charge for battery 402.

Ring 324 may be a visual indicator to notify the user of system 300 of different modes that system 300 is operating under and certain warnings to the user.

Computer subsystem 504 includes computer 408, display 304, and speaker 536. Computer 504 includes an operating system and software to operate system 300. Computer 504 may receive and process information from other components (for example, tracking subsystem 532, platform subsystem 502, and/or motion control subsystem 506) in order to display information to the user. Further, computer subsystem 504 may also include speaker 536 to provide audio to the user.

Tracking subsystem 532 may include position sensor 504 and converter 542. Tracking subsystem 532 may correspond to camera stand 302 including camera 326 as described with respect to FIG. 3. Position sensor 504 may be camera 326. Tracking subsystem may track the location of certain markers that are located on the different components of system 300 and/or instruments used by a user during a medical procedure. This tracking may be conducted in a manner consistent with the present disclosure including the use of infrared technology that tracks the location of active or passive elements, such as LEDs or reflective markers, respectively. The location, orientation, and position of structures having these types of markers may be provided to computer 408 which may be shown to a user on display 304. For example, a medical instrument 608 having these types of markers and tracked in this manner (which may be referred to as a navigational space) may be shown to a user in relation to a three dimensional image of a patient's anatomical structure.

Motion control subsystem 506 may be configured to physically move vertical column 312, upper arm 306, lower arm 308, or rotate end-effector 310. The physical movement may be conducted through the use of one or more motors 510-518. For example, motor 510 may be configured to vertically lift or lower vertical column 312. Motor 512 may be configured to laterally move upper arm 308 around a point of engagement with vertical column 312 as shown in FIG. 3. Motor 514 may be configured to laterally move lower arm 308 around a point of engagement with upper arm 308 as shown in FIG. 3. Motors 516 and 518 may be configured to move end-effector 310 in a manner such that one may control the roll and one may control the tilt, thereby providing multiple angles that end-effector 310 may be moved. These movements may be achieved by controller 538 which may control these movements through load cells disposed on end-effector 310 and activated by a user engaging these load cells to move system 300 in a desired manner.

Moreover, system 300 may provide for automatic movement of vertical column 312, upper arm 306, and lower arm 308 through a user indicating on display 304 (which may be a touchscreen input device) the location of a medical instrument or component on a three dimensional image of the patient's anatomy on display 304. The user may initiate this automatic movement by stepping on foot pedal 544 or some other input means.

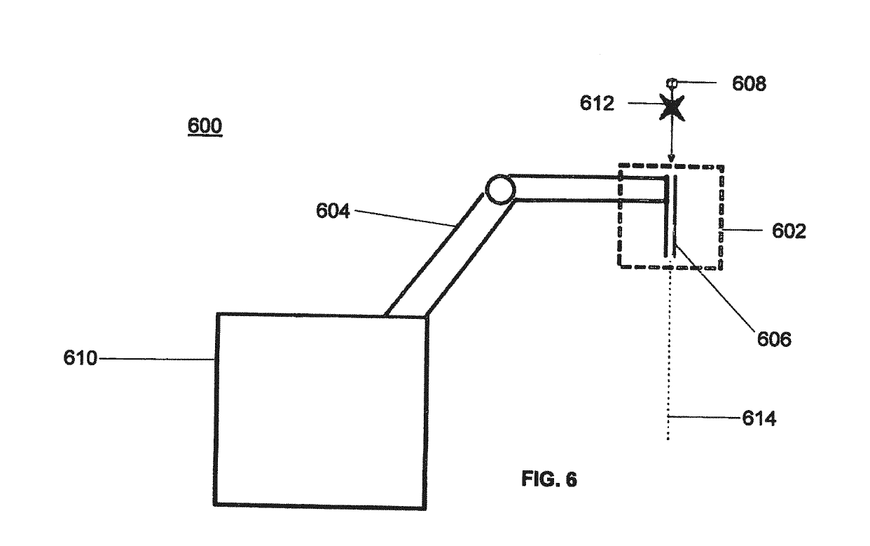

FIG. 6 illustrates a medical robot system 600 consistent with some embodiments. Medical robot system 600 may comprise end-effector 602, robot arm 604, guide tube 606, instrument 608, and robot base 610. Instrument tool 608 may be attached to a tracking array 612 including one or more tracking markers (such as markers 118) and have an associated trajectory 614. Trajectory 614 may represent a path of movement that instrument tool 608 is configured to travel once it is positioned through or secured in guide tube 606, for example, a path of insertion of instrument tool 608 into a patient. In an operation, robot base 610 may be configured to be in electronic communication with robot arm 604 and end-effector 602 so that medical robot system 600 may assist a user (for example, a doctor) in operating on the patient 210. Medical robot system 600 may be consistent with previously described medical robot system 100 and 300.

A tracking array 612 may be mounted on instrument 608 to monitor the location and orientation of instrument tool 608. The tracking array 612 may be attached to an instrument 608 and may comprise tracking markers 804. As best seen in FIG. 8, tracking markers 804 may be, for example, light emitting diodes and/or other types of reflective markers (e.g., markers 118 as described elsewhere herein). The tracking devices may be one or more line of sight devices associated with the medical robot system. As an example, the tracking devices may be one or more cameras 200, 326 associated with the medical robot system 100, 300 and may also track tracking array 612 for a defined domain or relative orientations of the instrument 608 in relation to the robot arm 604, the robot base 610, end-effector 602, and/or the patient 210. The tracking devices may be consistent with those structures described in connection with camera stand 302 and tracking subsystem 532.

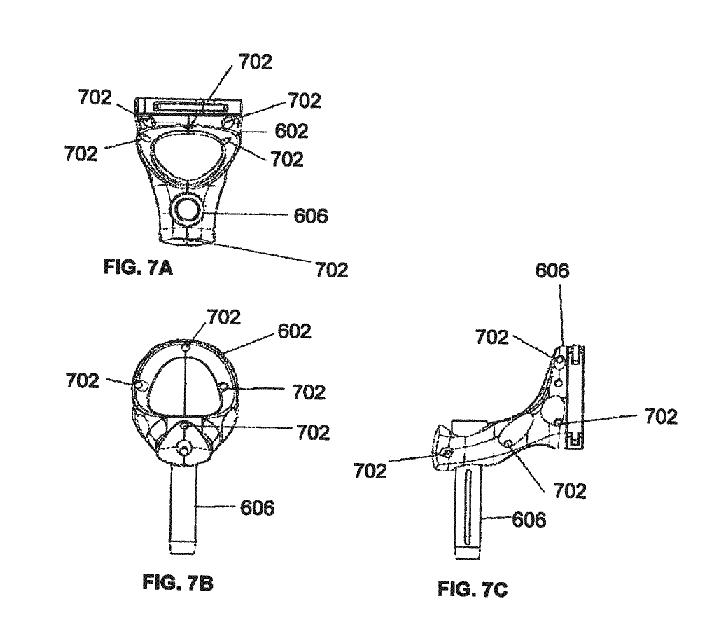

FIGS. 7A, 7B, and 7C illustrate a top view, front view, and side view, respectively, of end-effector 602 consistent with some embodiments. End-effector 602 may comprise one or more tracking markers 702. Tracking markers 702 may be light emitting diodes or other types of active and passive markers, such as tracking markers 118 that have been previously described. In some embodiments, the tracking markers 702 are active infrared-emitting markers that are activated by an electrical signal (e.g., infrared light emitting diodes (LEDs)). Thus, tracking markers 702 may be activated such that the infrared markers 702 are visible to the camera 200, 326 or may be deactivated such that the infrared markers 702 are not visible to the camera 200, 326. Thus, when the markers 702 are active, the end-effector 602 may be controlled by the system 100, 300, 600, and when the markers 702 are deactivated, the end-effector 602 may be locked in position and unable to be moved by the system 100, 300, 600.

Markers 702 may be disposed on or within end-effector 602 in a manner such that the markers 702 are visible by one or more cameras 200, 326 or other tracking devices associated with the medical robot system 100, 300, 600. The camera 200, 326 or other tracking devices may track end-effector 602 as it moves to different positions and viewing angles by following the movement of tracking markers 702. The location of markers 702 and/or end-effector 602 may be shown on a display 110, 304 associated with the medical robot system 100, 300, 600, for example, display 110 as shown in FIG. 2 and/or display 304 shown in FIG. 3. This display 110, 304 may allow a user to ensure that end-effector 602 is in a desirable position in relation to robot arm 604, robot base 610, the patient 210, and/or the user.

For example, as shown in FIG. 7A, markers 702 may be placed around the surface of end-effector 602 so that a tracking device placed away from the medical field 208 and facing toward the robot 102, 301 and the camera 200, 326 is able to view at least 3 of the markers 702 through a range of common orientations of the end-effector 602 relative to the tracking device. For example, distribution of markers 702 in this way allows end-effector 602 to be monitored by the tracking devices when end-effector 602 is translated and rotated in the medical field 208.

In addition, in some embodiments, end-effector 602 may be equipped with infrared (IR) receivers that can detect when an external camera 200, 326 is getting ready to read markers 702. Upon this detection, end-effector 602 may then illuminate markers 702. The detection by the IR receivers that the external camera 200, 326 is ready to read markers 702 may signal the need to synchronize a duty cycle of markers 702, which may be light emitting diodes, to an external camera 200, 326. This may also allow for lower power consumption by the robotic system as a whole, whereby markers 702 would only be illuminated at the appropriate time instead of being illuminated continuously. Further, in some embodiments, markers 702 may be powered off to prevent interference with other navigation tools, such as different types of medical instruments 608.

FIG. 8 depicts one type of medical instrument 608 including a tracking array 612 and tracking markers 804. Tracking markers 804 may be of any type described herein including but not limited to light emitting diodes or reflective spheres. Markers 804 are monitored by tracking devices associated with the medical robot system 100, 300, 600 and may be one or more of the line of sight cameras 200, 326. The cameras 200, 326 may track the location of instrument 608 based on the position and orientation of tracking array 612 and markers 804. A user, such as a doctor 120, may orient instrument 608 in a manner so that tracking array 612 and markers 804 are sufficiently recognized by the tracking device or camera 200, 326 to display instrument 608 and markers 804 on, for example, display 110 of the medical robot system.

The manner in which a doctor 120 may place instrument 608 into guide tube 606 of the end-effector 602 and adjust the instrument 608 is evident in FIG. 8. The hollow tube or guide tube 114, 606 of the end-effector 112, 310, 602 is sized and configured to receive at least a portion of the medical instrument 608. The guide tube 114, 606 is configured to be oriented by the robot arm 104 such that insertion and trajectory for the medical instrument 608 is able to reach a desired anatomical target within or upon the body of the patient 210. The medical instrument 608 may include at least a portion of a generally cylindrical instrument. Although a screw driver is exemplified as the medical tool 608, it will be appreciated that any suitable medical tool 608 may be positioned by the end-effector 602. By way of example, the medical instrument 608 may include one or more of a guide wire, cannula, a retractor, a drill, a reamer, a screw driver, an insertion tool, a removal tool, or the like. Although the hollow tube 114, 606 is generally shown as having a cylindrical configuration, it will be appreciated by those of skill in the art that the guide tube 114, 606 may have any suitable shape, size, and configuration desired to accommodate the medical instrument 608 and access the medical site.

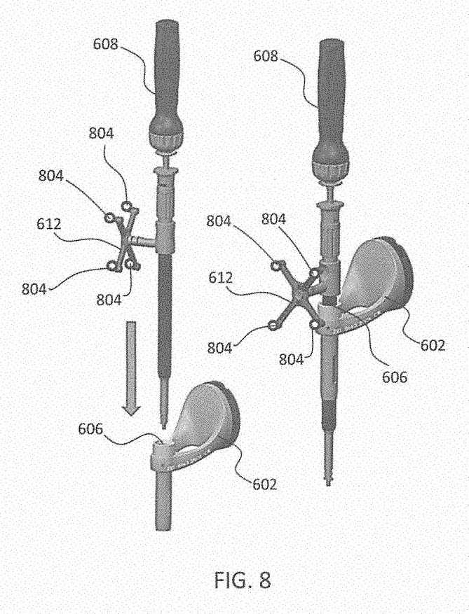

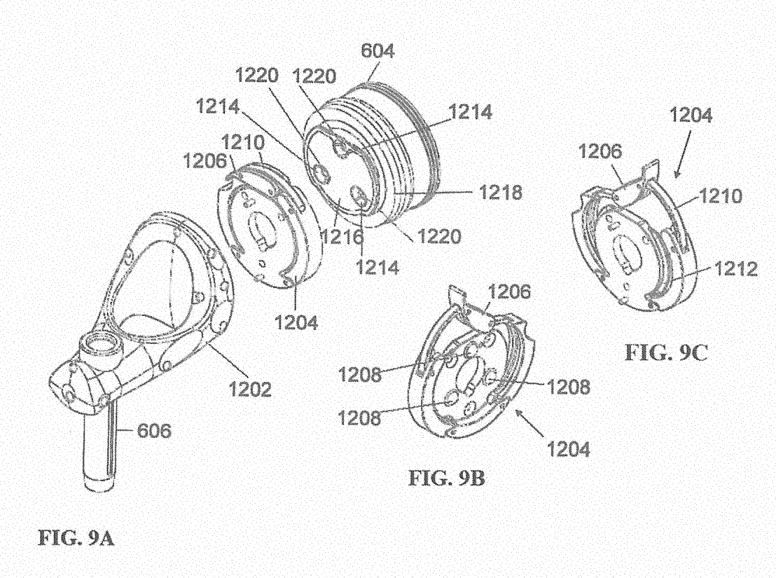

FIGS. 9A-9C illustrate end-effector 602 and a portion of robot arm 604 consistent with some embodiments. End-effector 602 may further comprise body 1202 and clamp 1204. Clamp 1204 may comprise handle 1206, balls 1208, spring 1210, and lip 1212. Robot arm 604 may further comprise depressions 1214, mounting plate 1216, lip 1218, and magnets 1220.

End-effector 602 may mechanically interface and/or engage with the medical robot system and robot arm 604 through one or more couplings. For example, end-effector 602 may engage with robot arm 604 through a locating coupling and/or a reinforcing coupling. Through these couplings, end-effector 602 may fasten with robot arm 604 outside a flexible and sterile barrier. In some embodiments, the locating coupling may be a magnetically kinematic mount and the reinforcing coupling may be a five bar over center clamping linkage.

With respect to the locating coupling, robot arm 604 may comprise mounting plate 1216, which may be non-magnetic material, one or more depressions 1214, lip 1218, and magnets 1220. Magnet 1220 is mounted below each of depressions 1214. Portions of clamp 1204 may comprise magnetic material and be attracted by one or more magnets 1220. Through the magnetic attraction of clamp 1204 and robot arm 604, balls 1208 become seated into respective depressions 1214. For example, balls 1208 as shown in FIG. 9B would be seated in depressions 1214 as shown in FIG. 9A. This seating may be considered a magnetically-assisted kinematic coupling. Magnets 1220 may be configured to be strong enough to support the entire weight of end-effector 602 regardless of the orientation of end-effector 602. The locating coupling may be any style of kinematic mount that uniquely restrains six degrees of freedom.

With respect to the reinforcing coupling, portions of clamp 1204 may be configured to be a fixed ground link and as such clamp 1204 may serve as a five bar linkage. Closing clamp handle 1206 may fasten end-effector 602 to robot arm 604 as lip 1212 and lip 1218 engage clamp 1204 in a manner to secure end-effector 602 and robot arm 604. When clamp handle 1206 is closed, spring 1210 may be stretched or stressed while clamp 1204 is in a locked position. The locked position may be a position that provides for linkage past center. Because of a closed position that is past center, the linkage will not open absent a force applied to clamp handle 1206 to release clamp 1204. Thus, in a locked position end-effector 602 may be robustly secured to robot arm 604.

Spring 1210 may be a curved beam in tension. Spring 1210 may be comprised of a material that exhibits high stiffness and high yield strain such as virgin PEEK (poly-ether-ether-ketone). The linkage between end-effector 602 and robot arm 604 may provide for a sterile barrier between end-effector 602 and robot arm 604 without impeding fastening of the two couplings.

The reinforcing coupling may be a linkage with multiple spring members. The reinforcing coupling may latch with a cam or friction based mechanism. The reinforcing coupling may also be a sufficiently powerful electromagnet that will support fastening end-effector 102 to robot arm 604. The reinforcing coupling may be a multi-piece collar completely separate from either end-effector 602 and/or robot arm 604 that slips over an interface between end-effector 602 and robot arm 604 and tightens with a screw mechanism, an over center linkage, or a cam mechanism.

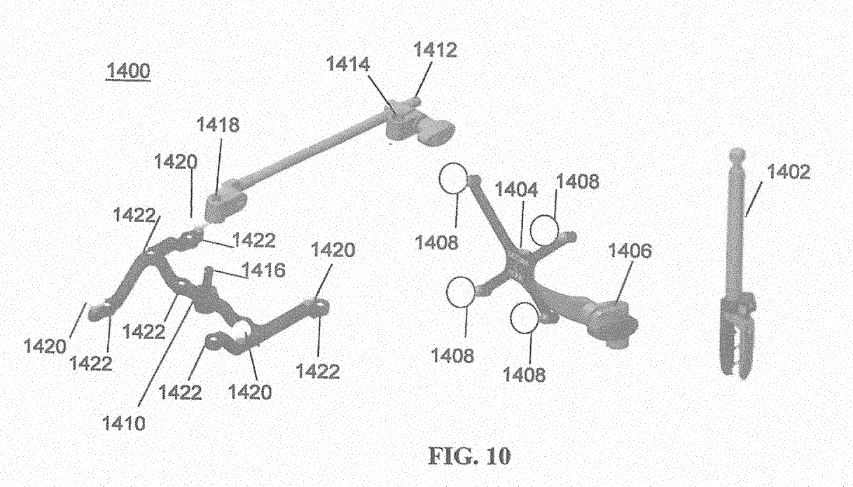

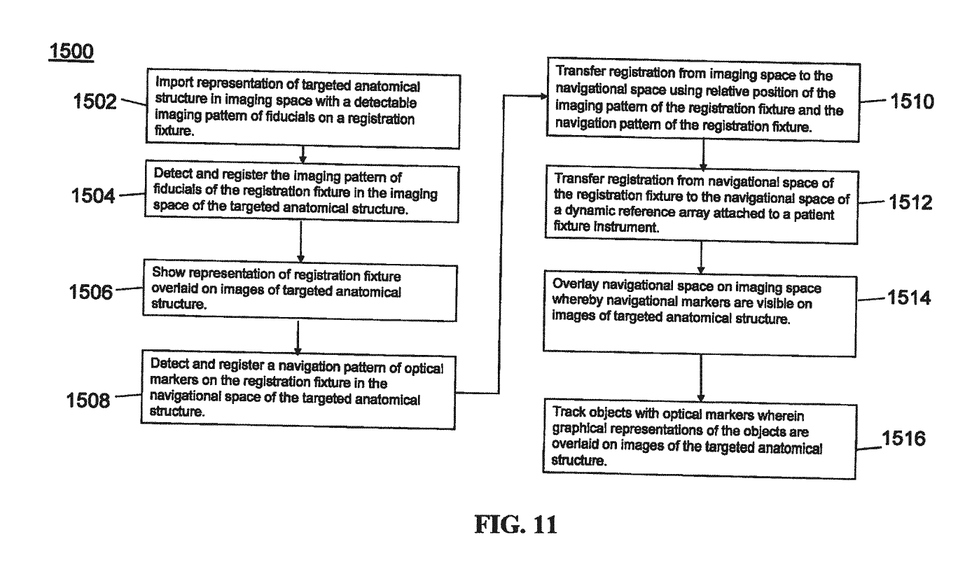

Referring to FIGS. 10 and 11, prior to or during a medical procedure, certain registration procedures may be conducted to track objects and a target anatomical structure of the patient 210 both in a navigation space and an image space. To conduct such registration, a registration system 1400 may be used as illustrated in FIG. 10.

To track the position of the patient 210, a patient tracking device 116 may include a patient fixation instrument 1402 to be secured to a rigid anatomical structure of the patient 210 and a dynamic reference base (DRB) 1404 may be securely attached to the patient fixation instrument 1402. For example, patient fixation instrument 1402 may be inserted into opening 1406 of dynamic reference base 1404. Dynamic reference base 1404 may contain markers 1408 that are visible to tracking devices, such as tracking subsystem 532. These markers 1408 may be optical markers or reflective spheres, such as tracking markers 118, as previously discussed herein.

Patient fixation instrument 1402 is attached to a rigid anatomy of the patient 210 and may remain attached throughout the medical procedure. In some embodiments, patient fixation instrument 1402 is attached to a rigid area of the patient 210, for example, a bone that is located away from the targeted anatomical structure subject to the medical procedure. In order to track the targeted anatomical structure, dynamic reference base 1404 is associated with the targeted anatomical structure through the use of a registration fixture that is temporarily placed on or near the targeted anatomical structure in order to register the dynamic reference base 1404 with the location of the targeted anatomical structure.

A registration fixture 1410 is attached to patient fixation instrument 1402 through the use of a pivot arm 1412. Pivot arm 1412 is attached to patient fixation instrument 1402 by inserting patient fixation instrument 1402 through an opening 1414 of registration fixture 1410. Pivot arm 1412 is attached to registration fixture 1410 by, for example, inserting a knob 1416 through an opening 1418 of pivot arm 1412.

Using pivot arm 1412, registration fixture 1410 may be placed over the targeted anatomical structure and its location may be determined in an image space and navigation space using tracking markers 1420 and/or fiducials 1422 on registration fixture 1410. Registration fixture 1410 may contain a collection of markers 1420 that are visible in a navigational space (for example, markers 1420 may be detectable by tracking subsystem 532). Tracking markers 1420 may be optical markers visible in infrared light as previously described herein. Registration fixture 1410 may also contain a collection of fiducials 1422, for example, such as bearing balls, that are visible in an imaging space (for example, a three dimension CT image). As described in greater detail with respect to FIG. 11, using registration fixture 1410, the targeted anatomical structure may be associated with dynamic reference base 1404 thereby allowing depictions of objects in the navigational space to be overlaid on images of the anatomical structure. Dynamic reference base 1404, located at a position away from the targeted anatomical structure, may become a reference point thereby allowing removal of registration fixture 1410 and/or pivot arm 1412 from the medical area.

FIG. 11 provides a method 1500 for registration consistent with the present disclosure. Method 1500 begins at step 1502 wherein a graphical representation (or image(s)) of the targeted anatomical structure may be imported into system 100, 300 600, for example computer 408. The graphical representation may be three dimensional CT or a fluoroscope scan of the targeted anatomical structure of the patient 210 which includes registration fixture 1410 and a detectable imaging pattern of fiducials 1420.

At step 1504, an imaging pattern of fiducials 1420 is detected and registered in the imaging space and stored in computer 408. Optionally, at this time at step 1506, a graphical representation of the registration fixture 1410 may be overlaid on the images of the targeted anatomical structure.

At step 1508, a navigational pattern of registration fixture 1410 is detected and registered by recognizing markers 1420. Markers 1420 may be optical markers that are recognized in the navigation space through infrared light by tracking subsystem 532 via position sensor 540. Thus, the location, orientation, and other information of the targeted anatomical structure is registered in the navigation space. Therefore, registration fixture 1410 may be recognized in both the image space through the use of fiducials 1422 and the navigation space through the use of markers 1420. At step 1510, the registration of registration fixture 1410 in the image space is transferred to the navigation space. This transferal is done, for example, by using the relative position of the imaging pattern of fiducials 1422 compared to the position of the navigation pattern of markers 1420.

At step 1512, registration of the navigation space of registration fixture 1410 (having been registered with the image space) is further transferred to the navigation space of dynamic registration array 1404 attached to patient fixture instrument 1402. Thus, registration fixture 1410 may be removed and dynamic reference base 1404 may be used to track the targeted anatomical structure in both the navigation and image space because the navigation space is associated with the image space.

At steps 1514 and 1516, the navigation space may be overlaid on the image space and objects with markers visible in the navigation space (for example, medical instruments 608 with optical markers 804). The objects may be tracked through graphical representations of the medical instrument 608 on the images of the targeted anatomical structure.

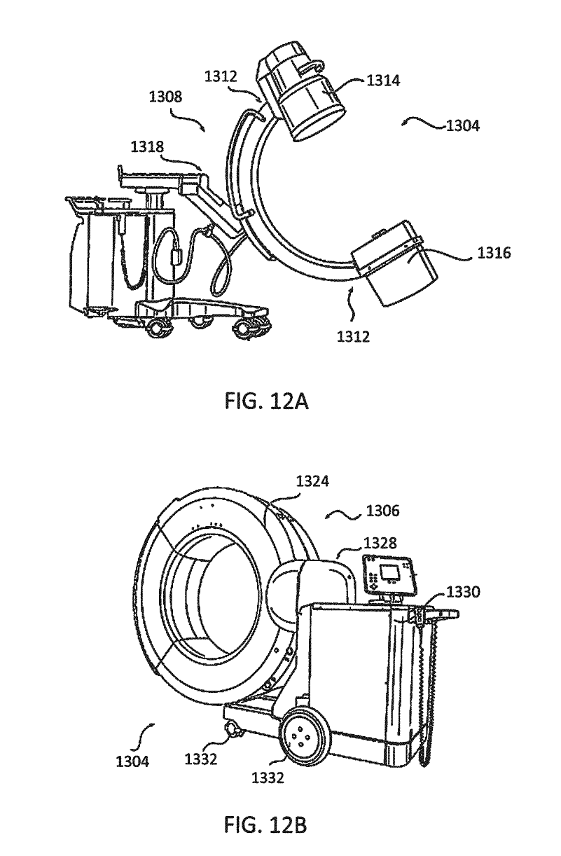

FIGS. 12A-12B illustrate imaging devices 1304 that may be used in conjunction with robot systems 100, 300, 600 to acquire pre-operative, intra-operative, post-operative, and/or real-time image data of patient 210. Any appropriate subject matter may be imaged for any appropriate procedure using the image-guided surgical system 1304 (also referred to as an imaging system). The image-guided surgical system 1304 may be any imaging device such as imaging device 1306 and/or a C-arm 1308 device. It may be desirable to take x-rays of patient 210 from a number of different positions, without the need for frequent manual repositioning of patient 210 which may be required in an x-ray system. As illustrated in FIG. 12A, the image-guided surgical system 1304 may be in the form of a C-arm 1308 that includes an elongated C-shaped member terminating in opposing distal ends 1312 of the “C” shape. C-shaped member 1130 may further comprise an x-ray source 1314 and an image receptor 1316. The space within C-arm 1308 of the arm may provide room for the physician to attend to the patient substantially free of interference from x-ray support structure 1318. As illustrated in FIG. 12B, the image-guided surgical system may include imaging device 1306 having a gantry housing 1324 attached to a support structure imaging device support structure 1328, such as a wheeled mobile cart 1330 with wheels 1332, which may enclose an image capturing portion, not illustrated. The image capturing portion may include an x-ray source and/or emission portion and an x-ray receiving and/or image receiving portion, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of patient 210 to be acquired from multiple directions or in multiple planes. Although certain image-guided surgical systems 1304 are exemplified herein, it will be appreciated that any suitable image-guided surgical system may be selected by one of ordinary skill in the art.

Turning now to FIGS. 13A-13C, the medical robot system 100, 300, 600 relies on accurate positioning of the end-effector 112, 602, medical instruments 608, and/or the patient 210 (e.g., patient tracking device 116) relative to the desired medical area. In the embodiments shown in FIGS. 13A-13C, the tracking markers 118, 804 are rigidly attached to a portion of the instrument 608 and/or end-effector 112.

FIG. 13A depicts part of the medical robot system 100 with the robot 102 including base 106, robot arm 104, and end-effector 112. The other elements, not illustrated, such as the display, cameras, etc. may also be present as described herein. FIG. 13B depicts a close-up view of the end-effector 112 with guide tube 114 and a plurality of tracking markers 118 rigidly affixed to the end-effector 112. In this embodiment, the plurality of tracking markers 118 are attached to the guide tube 112. FIG. 13C depicts an instrument 608 (in this case, a probe 608A) with a plurality of tracking markers 804 rigidly affixed to the instrument 608. As described elsewhere herein, the instrument 608 could include any suitable medical instrument, such as, but not limited to, guide wire, cannula, a retractor, a drill, a reamer, a screw driver, an insertion tool, a removal tool, or the like.

When tracking an instrument 608, end-effector 112, or other object to be tracked in 3D, an array of tracking markers 118, 804 may be rigidly attached to a portion of the tool 608 or end-effector 112. Preferably, the tracking markers 118, 804 are attached such that the markers 118, 804 are out of the way (e.g., not impeding the medical operation, visibility, etc.). The markers 118, 804 may be affixed to the instrument 608, end-effector 112, or other object to be tracked, for example, with an array 612. Usually three or four markers 118, 804 are used with an array 612. The array 612 may include a linear section, a cross piece, and may be asymmetric such that the markers 118, 804 are at different relative positions and locations with respect to one another. For example, as shown in FIG. 13C, a probe 608A with a 4-marker tracking array 612 is shown, and FIG. 13B depicts the end-effector 112 with a different 4-marker tracking array 612.

In FIG. 13C, the tracking array 612 functions as the handle 620 of the probe 608A. Thus, the four markers 804 are attached to the handle 620 of the probe 608A, which is out of the way of the shaft 622 and tip 624. Stereophotogrammetric tracking of these four markers 804 allows the instrument 608 to be tracked as a rigid body and for the tracking system 100, 300, 600 to precisely determine the position of the tip 624 and the orientation of the shaft 622 while the probe 608A is moved around in front of tracking cameras 200, 326.

To enable automatic tracking of one or more tools 608, end-effector 112, or other object to be tracked in 3D (e.g., multiple rigid bodies), the markers 118, 804 on each tool 608, end-effector 112, or the like, are arranged asymmetrically with a known inter-marker spacing. The reason for asymmetric alignment is so that it is unambiguous which marker 118, 804 corresponds to a particular location on the rigid body and whether markers 118, 804 are being viewed from the front or back, i.e., mirrored. For example, if the markers 118, 804 were arranged in a square on the tool 608 or end-effector 112, it would be unclear to the system 100, 300, 600 which markers 118, 804 corresponded to which corner of the square. For example, for the probe 608A, it would be unclear which marker 804 was closest to the shaft 622. Thus, it would be unknown which way the shaft 622 was extending from the array 612. Accordingly, each array 612 and thus each tool 608, end-effector 112, or other object to be tracked should have a unique marker pattern to allow it to be distinguished from other tools 608 or other objects being tracked. Asymmetry and unique marker patterns allow the system 100, 300, 600 to detect individual markers 118, 804 then to check the marker spacing against a stored template to determine which tool 608, end effector 112, or other object they represent. Detected markers 118, 804 can then be sorted automatically and assigned to each tracked object in the correct order. Without this information, rigid body calculations could not then be performed to extract key geometric information, for example, such as tool tip 624 and alignment of the shaft 622, unless the user manually specified which detected marker 118, 804 corresponded to which position on each rigid body. These concepts are commonly known to those skilled in the methods of 3D optical tracking.

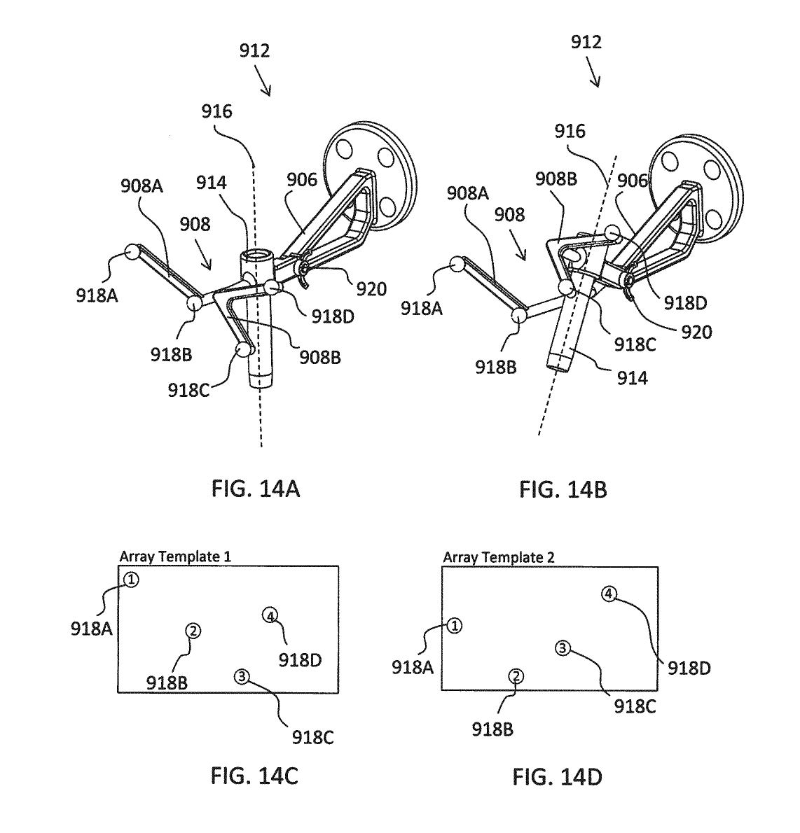

Turning now to FIGS. 14A-14D, an alternative version of an end-effector 912 with moveable tracking markers 918A-918D is shown. In FIG. 14A, an array with moveable tracking markers 918A-918D are shown in a first configuration, and in FIG. 14B the moveable tracking markers 918A-918D are shown in a second configuration, which is angled relative to the first configuration. FIG. 14C shows the template of the tracking markers 918A-918D, for example, as seen by the cameras 200, 326 in the first configuration of FIG. 14A; and FIG. 14D shows the template of tracking markers 918A-918D, for example, as seen by the cameras 200, 326 in the second configuration of FIG. 14B.

In this embodiment, 4-marker array tracking is contemplated wherein the markers 918A-918D are not all in fixed position relative to the rigid body and instead, one or more of the array markers 918A-918D can be adjusted, for example, during testing, to give updated information about the rigid body that is being tracked without disrupting the process for automatic detection and sorting of the tracked markers 918A-918D.

When tracking any tool, such as a guide tube 914 connected to the end effector 912 of a robot system 100, 300, 600, the tracking array's primary purpose is to update the position of the end effector 912 in the camera coordinate system. When using the rigid system, for example, as shown in FIG. 13B, the array 612 of reflective markers 118 rigidly extend from the guide tube 114. Because the tracking markers 118 are rigidly connected, knowledge of the marker locations in the camera coordinate system also provides exact location of the centerline, tip, and tail of the guide tube 114 in the camera coordinate system. Typically, information about the position of the end effector 112 from such an array 612 and information about the location of a target trajectory from another tracked source are used to calculate the required moves that must be input for each axis of the robot 102 that will move the guide tube 114 into alignment with the trajectory and move the tip to a particular location along the trajectory vector.

Sometimes, the desired trajectory is in an awkward or unreachable location, but if the guide tube 114 could be swiveled, it could be reached. For example, a very steep trajectory pointing away from the base 106 of the robot 102 might be reachable if the guide tube 114 could be swiveled upward beyond the limit of the pitch (wrist up-down angle) axis, but might not be reachable if the guide tube 114 is attached parallel to the plate connecting it to the end of the wrist. To reach such a trajectory, the base 106 of the robot 102 might be moved or a different end effector 112 with a different guide tube attachment might be exchanged with the working end effector. Both of these solutions may be time consuming and cumbersome.

As best seen in FIGS. 14A and 14B, if the array 908 is configured such that one or more of the markers 918A-918D are not in a fixed position and instead, one or more of the markers 918A-918D can be adjusted, swiveled, pivoted, or moved, the robot 102 can provide updated information about the object being tracked without disrupting the detection and tracking process. For example, one of the markers 918A-918D may be fixed in position and the other markers 918A-918D may be moveable; two of the markers 918A-918D may be fixed in position and the other markers 918A-918D may be moveable; three of the markers 918A-918D may be fixed in position and the other marker 918A-918D may be moveable; or all of the markers 918A-918D may be moveable.

In the embodiment shown in FIGS. 14A and 14B, markers 918A, 918 B are rigidly connected directly to a base 906 of the end-effector 912, and markers 918C, 918D are rigidly connected to the tube 914. Similar to array 612, array 908 may be provided to attach the markers 918A-918D to the end-effector 912, instrument 608, or other object to be tracked. In this case, however, the array 908 is comprised of a plurality of separate components. For example, markers 918A, 918B may be connected to the base 906 with a first array 908A, and markers 918C, 918D may be connected to the guide tube 914 with a second array 908B. Marker 918A may be affixed to a first end of the first array 908A and marker 918B may be separated a linear distance and affixed to a second end of the first array 908A. While first array 908 is substantially linear, second array 908B has a bent or V-shaped configuration, with respective root ends, connected to the guide tube 914, and diverging therefrom to distal ends in a V-shape with marker 918C at one distal end and marker 918D at the other distal end. Although specific configurations are exemplified herein, it will be appreciated that other asymmetric designs including different numbers and types of arrays 908A, 908B and different arrangements, numbers, and types of markers 918A-918D are contemplated.

The guide tube 914 may be moveable, swivelable, or pivotable relative to the base 906, for example, across a hinge 920 or other connector to the base 906. Thus, markers 918C, 918D are moveable such that when the guide tube 914 pivots, swivels, or moves, markers 918C, 918D also pivot, swivel, or move. As best seen in FIG. 14A, guide tube 914 has a longitudinal axis 916 which is aligned in a substantially normal or vertical orientation such that markers 918A-918D have a first configuration. Turning now to FIG. 14B, the guide tube 914 is pivoted, swiveled, or moved such that the longitudinal axis 916 is now angled relative to the vertical orientation such that markers 918A-918D have a second configuration, different from the first configuration.

In contrast to the embodiment described for FIGS. 14A-14D, if a swivel existed between the guide tube 914 and the arm 104 (e.g., the wrist attachment) with all four markers 918A-918D remaining attached rigidly to the guide tube 914 and this swivel was adjusted by the user, the robotic system 100, 300, 600 would not be able to automatically detect that the guide tube 914 orientation had changed. The robotic system 100, 300, 600 would track the positions of the marker array 908 and would calculate incorrect robot axis moves assuming the guide tube 914 was attached to the wrist (the robot arm 104) in the previous orientation. By keeping one or more markers 918A-918D (e.g., two markers 918C, 918D) rigidly on the tube 914 and one or more markers 918A-918D (e.g., two markers 918A, 918B) across the swivel, automatic detection of the new position becomes possible and correct robot moves are calculated based on the detection of a new tool or end-effector 112, 912 on the end of the robot arm 104.

One or more of the markers 918A-918D are configured to be moved, pivoted, swiveled, or the like according to any suitable means. For example, the markers 918A-918D may be moved by a hinge 920, such as a clamp, spring, lever, slide, toggle, or the like, or any other suitable mechanism for moving the markers 918A-918D individually or in combination, moving the arrays 908A, 908B individually or in combination, moving any portion of the end-effector 912 relative to another portion, or moving any portion of the tool 608 relative to another portion.

As shown in FIGS. 14A and 14B, the array 908 and guide tube 914 may become reconfigurable by simply loosening the clamp or hinge 920, moving part of the array 908A, 908B relative to the other part 908A, 908B, and retightening the hinge 920 such that the guide tube 914 is oriented in a different position. For example, two markers 918C, 918D may be rigidly interconnected with the tube 914 and two markers 918A, 918B may be rigidly interconnected across the hinge 920 to the base 906 of the end-effector 912 that attaches to the robot arm 104. The hinge 920 may be in the form of a clamp, such as a wing nut or the like, which can be loosened and retightened to allow the user to quickly switch between the first configuration (FIG. 14A) and the second configuration (FIG. 14B).

The cameras 200, 326 detect the markers 918A-918D, for example, in one of the templates identified in FIGS. 14C and 14D. If the array 908 is in the first configuration (FIG. 14A) and tracking cameras 200, 326 detect the markers 918A-918D, then the tracked markers match Array Template 1 as shown in FIG. 14C. If the array 908 is the second configuration (FIG. 14B) and tracking cameras 200, 326 detect the same markers 918A-918D, then the tracked markers match Array Template 2 as shown in FIG. 14D. Array Template 1 and Array Template 2 are recognized by the system 100, 300, 600 as two distinct tools, each with its own uniquely defined spatial relationship between guide tube 914, markers 918A-918D, and robot attachment. The user could therefore adjust the position of the end-effector 912 between the first and second configurations without notifying the system 100, 300, 600 of the change and the system 100, 300, 600 would appropriately adjust the movements of the robot 102 to stay on trajectory.

In this embodiment, there are two assembly positions in which the marker array matches unique templates that allow the system 100, 300, 600 to recognize the assembly as two different tools or two different end effectors. In any position of the swivel between or outside of these two positions (namely, Array Template 1 and Array Template 2 shown in FIGS. 14C and 14D, respectively), the markers 918A-918D would not match any template and the system 100, 300, 600 would not detect any array present despite individual markers 918A-918D being detected by cameras 200, 326, with the result being the same as if the markers 918A-918D were temporarily blocked from view of the cameras 200, 326. It will be appreciated that other array templates may exist for other configurations, for example, identifying different instruments 608 or other end-effectors 112, 912, etc.

In the embodiment described, two discrete assembly positions are shown in FIGS. 14A and 14B. It will be appreciated, however, that there could be multiple discrete positions on a swivel joint, linear joint, combination of swivel and linear joints, pegboard, or other assembly where unique marker templates may be created by adjusting the position of one or more markers 918A-918D of the array relative to the others, with each discrete position matching a particular template and defining a unique tool 608 or end-effector 112, 912 with different known attributes. In addition, although exemplified for end effector 912, it will be appreciated that moveable and fixed markers 918A-918D may be used with any suitable instrument 608 or other object to be tracked.

When using an external 3D tracking system 100, 300, 600 to track a full rigid body array of three or more markers attached to a robot's end effector 112 (for example, as depicted in FIGS. 13A and 13B), it is possible to directly track or to calculate the 3D position of every section of the robot 102 in the coordinate system of the cameras 200, 326. The geometric orientations of joints relative to the tracker are known by design, and the linear or angular positions of joints are known from encoders for each motor of the robot 102, fully defining the 3D positions of all of the moving parts from the end effector 112 to the base 116. Similarly, if a tracker were mounted on the base 106 of the robot 102 (not shown), it is likewise possible to track or calculate the 3D position of every section of the robot 102 from base 106 to end effector 112 based on known joint geometry and joint positions from each motor's encoder.

In some situations, it may be desirable to track the positions of all segments of the robot 102 from fewer than three markers 118 rigidly attached to the end effector 112. Specifically, if a tool 608 is introduced into the guide tube 114, it may be desirable to track full rigid body motion of the robot 902 with only one additional marker 118 being tracked.

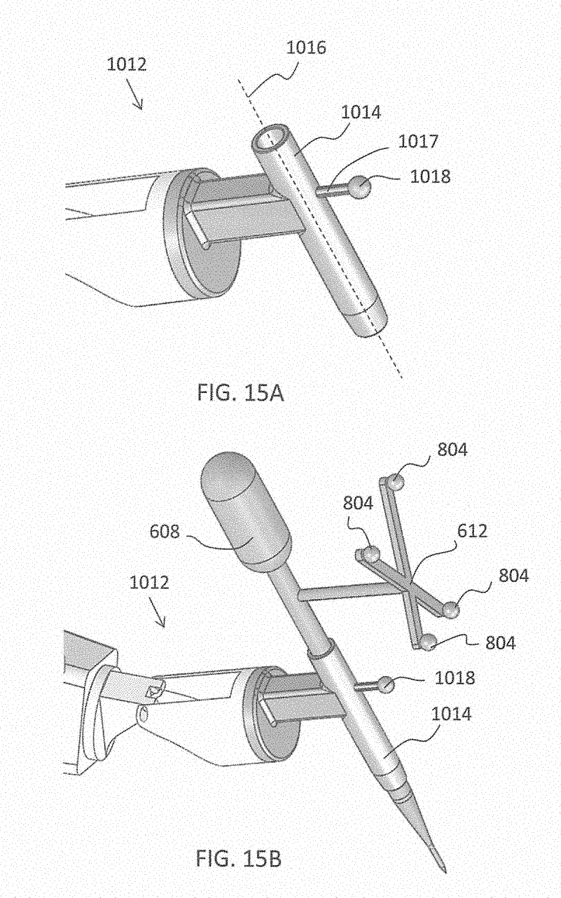

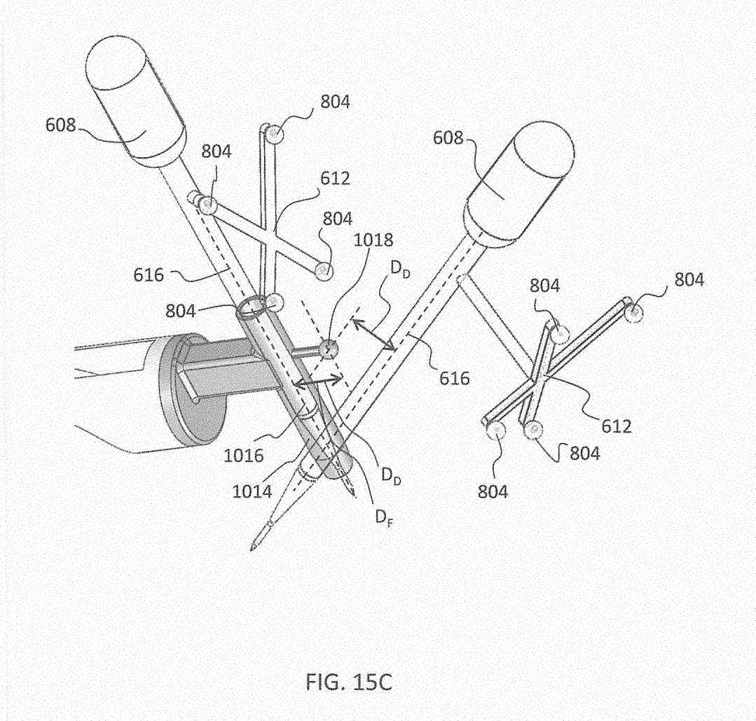

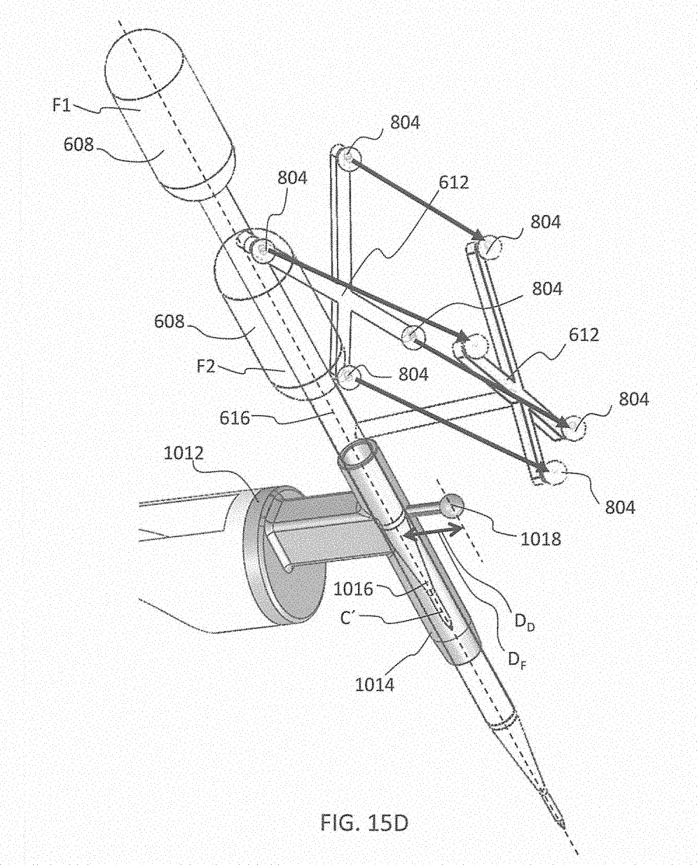

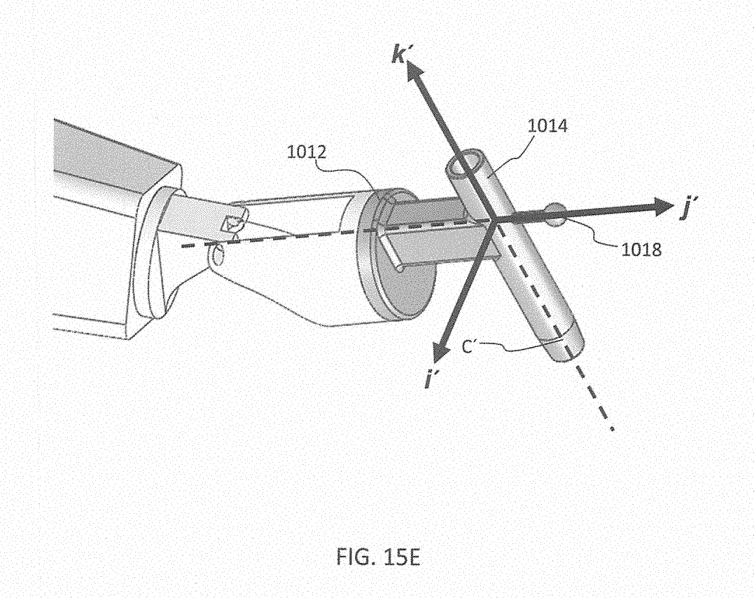

Turning now to FIGS. 15A-15E, an alternative version of an end-effector 1012 having only a single tracking marker 1018 is shown. End-effector 1012 may be similar to the other end-effectors described herein, and may include a guide tube 1014 extending along a longitudinal axis 1016. A single tracking marker 1018, similar to the other tracking markers described herein, may be rigidly affixed to the guide tube 1014. This single marker 1018 can serve the purpose of adding missing degrees of freedom to allow full rigid body tracking and/or can serve the purpose of acting as a surveillance marker to ensure that assumptions about robot and camera positioning are valid.

The single tracking marker 1018 may be attached to the robotic end effector 1012 as a rigid extension to the end effector 1012 that protrudes in any convenient direction and does not obstruct the doctor's view. The tracking marker 1018 may be affixed to the guide tube 1014 or any other suitable location of on the end-effector 1012. When affixed to the guide tube 1014, the tracking marker 1018 may be positioned at a location between first and second ends of the guide tube 1014. For example, in FIG. 15A, the single tracking marker 1018 is shown as a reflective sphere mounted on the end of a narrow shaft 1017 that extends forward from the guide tube 1014 and is positioned longitudinally above a mid-point of the guide tube 1014 and below the entry of the guide tube 1014. This position allows the marker 1018 to be generally visible by cameras 200, 326 but also would not obstruct vision of the doctor 120 or collide with other tools or objects in the vicinity of surgery. In addition, the guide tube 1014 with the marker 1018 in this position is designed for the marker array on any tool 608 introduced into the guide tube 1014 to be visible at the same time as the single marker 1018 on the guide tube 1014 is visible.

As shown in FIG. 15B, when a snugly fitting tool or instrument 608 is placed within the guide tube 1014, the instrument 608 becomes mechanically constrained in 4 of 6 degrees of freedom. That is, the instrument 608 cannot be rotated in any direction except about the longitudinal axis 1016 of the guide tube 1014 and the instrument 608 cannot be translated in any direction except along the longitudinal axis 1016 of the guide tube 1014. In other words, the instrument 608 can only be translated along and rotated about the centerline of the guide tube 1014. If two more parameters are known, such as (1) an angle of rotation about the longitudinal axis 1016 of the guide tube 1014; and (2) a position along the guide tube 1014, then the position of the end effector 1012 in the camera coordinate system becomes fully defined.

Referring now to FIG. 15C, the system 100, 300, 600 should be able to know when a tool 608 is actually positioned inside of the guide tube 1014 and is not instead outside of the guide tube 1014 and just somewhere in view of the cameras 200, 326. The tool 608 has a longitudinal axis or centerline 616 and an array 612 with a plurality of tracked markers 804. The rigid body calculations may be used to determine where the centerline 616 of the tool 608 is located in the camera coordinate system based on the tracked position of the array 612 on the tool 608.

The fixed normal (perpendicular) distance D_{F} from the single marker 1018 to the centerline or longitudinal axis 1016 of the guide tube 1014 is fixed and is known geometrically, and the position of the single marker 1018 can be tracked. Therefore, when a detected distance D_{D} from tool centerline 616 to single marker 1018 matches the known fixed distance D_{F} from the guide tube centerline 1016 to the single marker 1018, it can be determined that the tool 608 is either within the guide tube 1014 (centerlines 616, 1016 of tool 608 and guide tube 1014 coincident) or happens to be at some point in the locus of possible positions where this distance D_{D} matches the fixed distance D_{F}. For example, in FIG. 15C, the normal detected distance D_{D} from tool centerline 616 to the single marker 1018 matches the fixed distance D_{F} from guide tube centerline 1016 to the single marker 1018 in both frames of data (tracked marker coordinates) represented by the transparent tool 608 in two positions, and thus, additional considerations may be needed to determine when the tool 608 is located in the guide tube 1014.

Turning now to FIG. 15D, programmed logic can be used to look for frames of tracking data in which the detected distance D_{D} from tool centerline 616 to single marker 1018 remains fixed at the correct length despite the tool 608 moving in space by more than some minimum distance relative to the single sphere 1018 to satisfy the condition that the tool 608 is moving within the guide tube 1014. For example, a first frame F1 may be detected with the tool 608 in a first position and a second frame F2 may be detected with the tool 608 in a second position (namely, moved linearly with respect to the first position). The markers 804 on the tool array 612 may move by more than a given amount (e.g., more than 5 mm total) from the first frame F1 to the second frame F2. Even with this movement, the detected distance D_{D} from the tool centerline vector C′ to the single marker 1018 is substantially identical in both the first frame F1 and the second frame F2.

Logistically, the doctor 120 or user could place the tool 608 within the guide tube 1014 and slightly rotate it or slide it down into the guide tube 1014 and the system 100, 300, 600 would be able to detect that the tool 608 is within the guide tube 1014 from tracking of the five markers (four markers 804 on tool 608 plus single marker 1018 on guide tube 1014). Knowing that the tool 608 is within the guide tube 1014, all 6 degrees of freedom may be calculated that define the position and orientation of the robotic end effector 1012 in space. Without the single marker 1018, even if it is known with certainty that the tool 608 is within the guide tube 1014, it is unknown where the guide tube 1014 is located along the tool's centerline vector C′ and how the guide tube 1014 is rotated relative to the centerline vector C′.

With emphasis on FIG. 15E, the presence of the single marker 1018 being tracked as well as the four markers 804 on the tool 608, it is possible to construct the centerline vector C′ of the guide tube 1014 and tool 608 and the normal vector through the single marker 1018 and through the centerline vector C′. This normal vector has an orientation that is in a known orientation relative to the forearm of the robot distal to the wrist (in this example, oriented parallel to that segment) and intersects the centerline vector C′ at a specific fixed position. For convenience, three mutually orthogonal vectors k′, j′, i′ can be constructed, as shown in FIG. 15E, defining rigid body position and orientation of the guide tube 1014. One of the three mutually orthogonal vectors k′ is constructed from the centerline vector C′, the second vector j is constructed from the normal vector through the single marker 1018, and the third vector is the vector cross product of the first and second vectors k′, j′. The robot's joint positions relative to these vectors k′, j′, i′ are known and fixed when all joints are at zero, and therefore rigid body calculations can be used to determine the location of any section of the robot relative to these vectors k′, j′, i′ when the robot is at a home position. During robot movement, if the positions of the tool markers 804 (while the tool 608 is in the guide tube 1014) and the position of the single marker 1018 are detected from the tracking system, and angles/linear positions of each joint are known from encoders, then position and orientation of any section of the robot can be determined.

In some embodiments, it may be useful to fix the orientation of the tool 608 relative to the guide tube 1014. For example, the end effector guide tube 1014 may be oriented in a particular position about its axis 1016 to allow machining or implant positioning. Although the orientation of anything attached to the tool 608 inserted into the guide tube 1014 is known from the tracked markers 804 on the tool 608, the rotational orientation of the guide tube 1014 itself in the camera coordinate system is unknown without the additional tracking marker 1018 (or multiple tracking markers in other embodiments) on the guide tube 1014. This marker 1018 provides essentially a “clock position” from −180° to +180° based on the orientation of the marker 1018 relative to the centerline vector C′. Thus, the single marker 1018 can provide additional degrees of freedom to allow full rigid body tracking and/or can act as a surveillance marker to ensure that assumptions about the robot and camera positioning are valid.

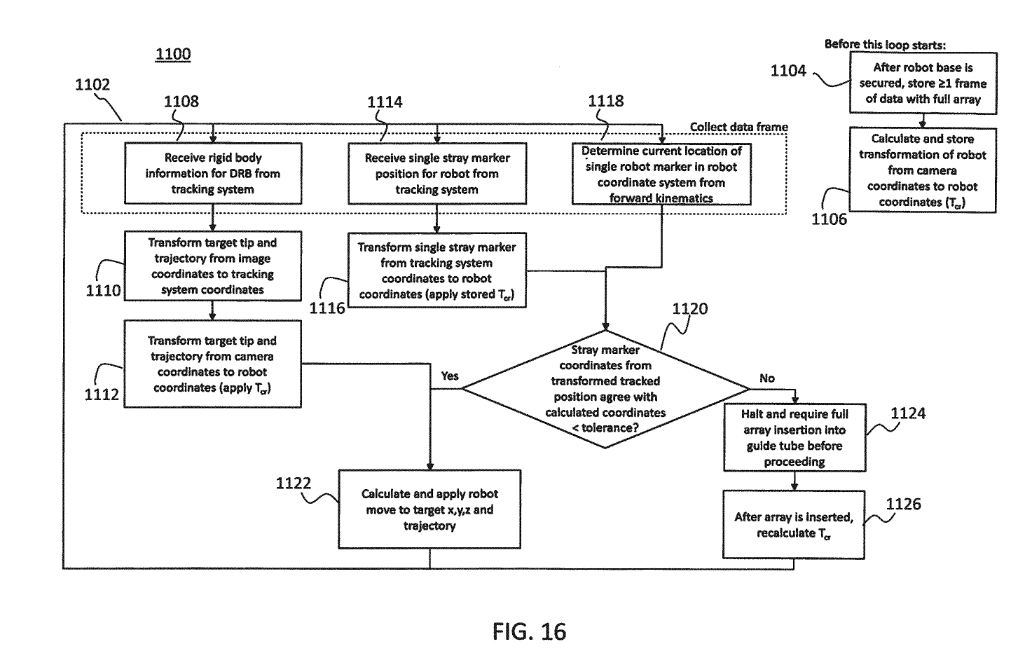

FIG. 16 is a block diagram of a method 1100 for navigating and moving the end-effector 1012 (or any other end-effector described herein) of the robot 102 to a desired target trajectory. Another use of the single marker 1018 on the robotic end effector 1012 or guide tube 1014 is as part of the method 1100 enabling the automated safe movement of the robot 102 without a full tracking array attached to the robot 102. This method 1100 functions when the tracking cameras 200, 326 do not move relative to the robot 102 (i.e., they are in a fixed position), the tracking system's coordinate system and robot's coordinate system are co-registered, and the robot 102 is calibrated such that the position and orientation of the guide tube 1014 can be accurately determined in the robot's Cartesian coordinate system based only on the encoded positions of each robotic axis.

For this method 1100, the coordinate systems of the tracker and the robot must be co-registered, meaning that the coordinate transformation from the tracking system's Cartesian coordinate system to the robot's Cartesian coordinate system is needed. For convenience, this coordinate transformation can be a 4×4 matrix of translations and rotations that is well known in the field of robotics. This transformation will be termed Tcr to refer to “transformation—camera to robot”. Once this transformation is known, any new frame of tracking data, which is received as x,y,z coordinates in vector form for each tracked marker, can be multiplied by the 4×4 matrix and the resulting x,y,z coordinates will be in the robot's coordinate system. To obtain Tcr, a full tracking array on the robot is tracked while it is rigidly attached to the robot at a location that is known in the robot's coordinate system, then known rigid body methods are used to calculate the transformation of coordinates. It should be evident that any tool 608 inserted into the guide tube 1014 of the robot 102 can provide the same rigid body information as a rigidly attached array when the additional marker 1018 is also read. That is, the tool 608 need only be inserted to any position within the guide tube 1014 and at any rotation within the guide tube 1014, not to a fixed position and orientation. Thus, it is possible to determine Tcr by inserting any tool 608 with a tracking array 612 into the guide tube 1014 and reading the tool's array 612 plus the single marker 1018 of the guide tube 1014 while at the same time determining from the encoders on each axis the current location of the guide tube 1014 in the robot's coordinate system.

Logic for navigating and moving the robot 102 to a target trajectory is provided in the method 1100 of FIG. 16. Before entering the loop 1102, it is assumed that the transformation Tcr was previously stored. Thus, before entering loop 1102, in step 1104, after the robot base 106 is secured, greater than or equal to one frame of tracking data of a tool inserted in the guide tube while the robot is static is stored; and in step 1106, the transformation of robot guide tube position from camera coordinates to robot coordinates Tcr is calculated from this static data and previous calibration data. Tcr should remain valid as long as the cameras 200, 326 do not move relative to the robot 102. If the cameras 200, 326 move relative to the robot 102, and Tcr needs to be re-obtained, the system 100, 300, 600 can be made to prompt the user to insert a tool 608 into the guide tube 1014 and then automatically perform the necessary calculations.

In the flowchart of method 1100, each frame of data collected consists of the tracked position of the DRB 1404 on the patient 210, the tracked position of the single marker 1018 on the end effector 1014, and a snapshot of the positions of each robotic axis. From the positions of the robot's axes, the location of the single marker 1018 on the end effector 1012 is calculated. This calculated position is compared to the actual position of the marker 1018 as recorded from the tracking system. If the values agree, it can be assured that the robot 102 is in a known location. The transformation Tcr is applied to the tracked position of the DRB 1404 so that the target for the robot 102 can be provided in terms of the robot's coordinate system. The robot 102 can then be commanded to move to reach the target.

After steps 1104, 1106, loop 1102 includes step 1108 receiving rigid body information for DRB 1404 from the tracking system; step 1110 transforming target tip and trajectory from image coordinates to tracking system coordinates; and step 1112 transforming target tip and trajectory from camera coordinates to robot coordinates (apply Tcr). Loop 1102 further includes step 1114 receiving a single stray marker position for robot from tracking system; and step 1116 transforming the single stray marker from tracking system coordinates to robot coordinates (apply stored Tcr). Loop 1102 also includes step 1118 determining current location of the single robot marker 1018 in the robot coordinate system from forward kinematics. The information from steps 1116 and 1118 is used to determine step 1120 whether the stray marker coordinates from transformed tracked position agree with the calculated coordinates being less than a given tolerance. If yes, proceed to step 1122, calculate and apply robot move to target x, y, z and trajectory. If no, proceed to step 1124, halt and require full array insertion into guide tube 1014 before proceeding; step 1126 after array is inserted, recalculate Tcr; and then proceed to repeat steps 1108, 1114, and 1118.

This method 1100 has advantages over a method in which the continuous monitoring of the single marker 1018 to verify the location is omitted. Without the single marker 1018, it would still be possible to determine the position of the end effector 1012 using Tcr and to send the end-effector 1012 to a target location but it would not be possible to verify that the robot 102 was actually in the expected location. For example, if the cameras 200, 326 had been bumped and Tcr was no longer valid, the robot 102 would move to an erroneous location. For this reason, the single marker 1018 provides value with regard to safety.

For a given fixed position of the robot 102, it is theoretically possible to move the tracking cameras 200, 326 to a new location in which the single tracked marker 1018 remains unmoved since it is a single point, not an array. In such a case, the system 100, 300, 600 would not detect any error since there would be agreement in the calculated and tracked locations of the single marker 1018. However, once the robot's axes caused the guide tube 1012 to move to a new location, the calculated and tracked positions would disagree and the safety check would be effective.

The term “surveillance marker” may be used, for example, in reference to a single marker that is in a fixed location relative to the DRB 1404. In this instance, if the DRB 1404 is bumped or otherwise dislodged, the relative location of the surveillance marker changes and the doctor 120 can be alerted that there may be a problem with navigation. Similarly, in the embodiments described herein, with a single marker 1018 on the robot's guide tube 1014, the system 100, 300, 600 can continuously check whether the cameras 200, 326 have moved relative to the robot 102. If registration of the tracking system's coordinate system to the robot's coordinate system is lost, such as by cameras 200, 326 being bumped or malfunctioning or by the robot malfunctioning, the system 100, 300, 600 can alert the user and corrections can be made. Thus, this single marker 1018 can also be thought of as a surveillance marker for the robot 102.

It should be clear that with a full array permanently mounted on the robot 102 (e.g., the plurality of tracking markers 702 on end-effector 602 shown in FIGS. 7A-7C) such functionality of a single marker 1018 as a robot surveillance marker is not needed because it is not required that the cameras 200, 326 be in a fixed position relative to the robot 102, and Tcr is updated at each frame based on the tracked position of the robot 102. Reasons to use a single marker 1018 instead of a full array are that the full array is more bulky and obtrusive, thereby blocking the doctor's view and access to the medical field 208 more than a single marker 1018, and line of sight to a full array is more easily blocked than line of sight to a single marker 1018.

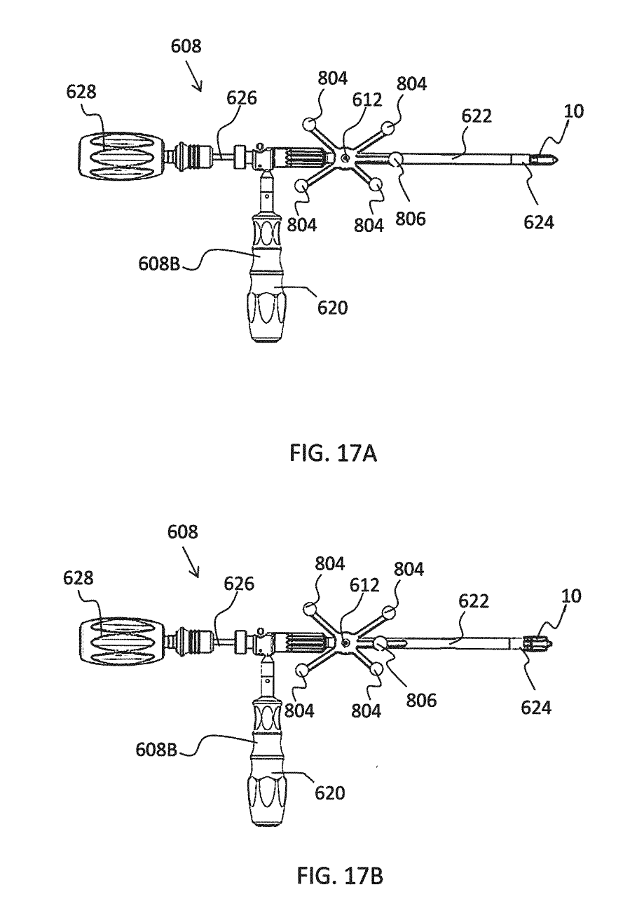

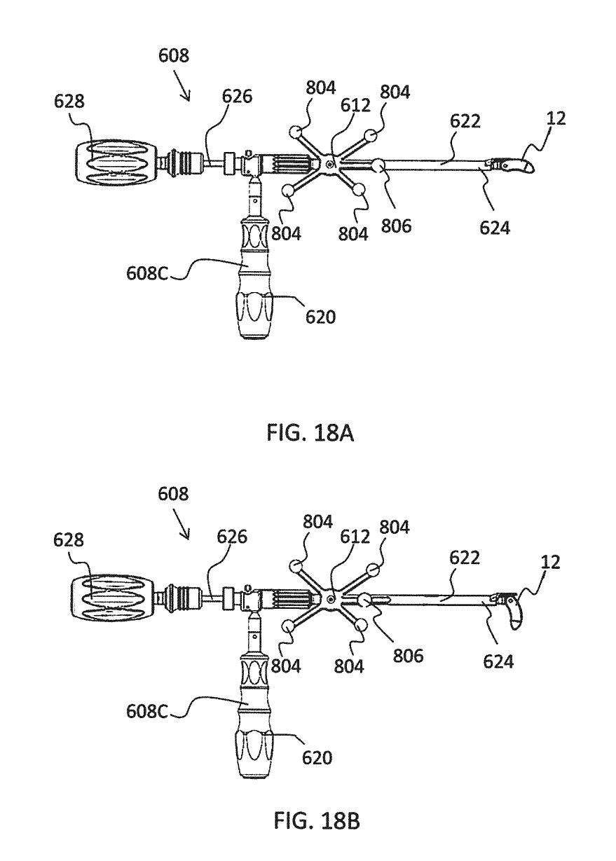

Turning now to FIGS. 17A-17B and 18A-18B, instruments 608, such as implant holders 608B, 608C, are depicted which include both fixed and moveable tracking markers 804, 806. The implant holders 608B, 608C may have a handle 620 and an outer shaft 622 extending from the handle 620. The shaft 622 may be positioned substantially perpendicular to the handle 620, as shown, or in any other suitable orientation. An inner shaft 626 may extend through the outer shaft 622 with a knob 628 at one end. Implant 10, 12 connects to the shaft 622, at the other end, at tip 624 of the implant holder 608B, 608C using typical connection mechanisms known to those of skill in the art. The knob 628 may be rotated, for example, to expand or articulate the implant 10, 12. U.S. Pat. Nos. 8,709,086 and 8,491,659, which are incorporated by reference herein, describe expandable fusion devices and methods of installation.

When tracking the tool 608, such as implant holder 608B, 608C, the tracking array 612 may contain a combination of fixed markers 804 and one or more moveable markers 806 which make up the array 612 or is otherwise attached to the implant holder 608B, 608C. The navigation array 612 may include at least one or more (e.g., at least two) fixed position markers 804, which are positioned with a known location relative to the implant holder instrument 608B, 608C. These fixed markers 804 would not be able to move in any orientation relative to the instrument geometry and would be useful in defining where the instrument 608 is in space. In addition, at least one marker 806 is present which can be attached to the array 612 or the instrument itself which is capable of moving within a pre-determined boundary (e.g., sliding, rotating, etc.) relative to the fixed markers 804. The system 100, 300, 600 (e.g., the software) correlates the position of the moveable marker 806 to a particular position, orientation, or other attribute of the implant 10 (such as height of an expandable interbody spacer shown in FIGS. 17A-17B or angle of an articulating interbody spacer shown in FIGS. 18A-18B). Thus, the system and/or the user can determine the height or angle of the implant 10, 12 based on the location of the moveable marker 806.

In the embodiment shown in FIGS. 17A-17B, four fixed markers 804 are used to define the implant holder 608B and a fifth moveable marker 806 is able to slide within a pre-determined path to provide feedback on the implant height (e.g., a contracted position or an expanded position). FIG. 17A shows the expandable spacer 10 at its initial height, and FIG. 17B shows the spacer 10 in the expanded state with the moveable marker 806 translated to a different position. In this case, the moveable marker 806 moves closer to the fixed markers 804 when the implant 10 is expanded, although it is contemplated that this movement may be reversed or otherwise different. The amount of linear translation of the marker 806 would correspond to the height of the implant 10. Although only two positions are shown, it would be possible to have this as a continuous function whereby any given expansion height could be correlated to a specific position of the moveable marker 806.

Turning now to FIGS. 18A-18B, four fixed markers 804 are used to define the implant holder 608C and a fifth, moveable marker 806 is configured to slide within a pre-determined path to provide feedback on the implant articulation angle. FIG. 18A shows the articulating spacer 12 at its initial linear state, and FIG. 18B shows the spacer 12 in an articulated state at some offset angle with the moveable marker 806 translated to a different position. The amount of linear translation of the marker 806 would correspond to the articulation angle of the implant 12. Although only two positions are shown, it would be possible to have this as a continuous function whereby any given articulation angle could be correlated to a specific position of the moveable marker 806.

In these embodiments, the moveable marker 806 slides continuously to provide feedback about an attribute of the implant 10, 12 based on position. It is also contemplated that there may be discreet positions that the moveable marker 806 must be in which would also be able to provide further information about an implant attribute. In this case, each discreet configuration of all markers 804, 806 correlates to a specific geometry of the implant holder 608B, 608C and the implant 10, 12 in a specific orientation or at a specific height. In addition, any motion of the moveable marker 806 could be used for other variable attributes of any other type of navigated implant.

Although depicted and described with respect to linear movement of the moveable marker 806, the moveable marker 806 should not be limited to just sliding as there may be applications where rotation of the marker 806 or other movements could be useful to provide information about the implant 10, 12. Any relative change in position between the set of fixed markers 804 and the moveable marker 806 could be relevant information for the implant 10, 12 or other device. In addition, although expandable and articulating implants 10, 12 are exemplified, the instrument 608 could work with other medical devices and materials, such as spacers, cages, plates, fasteners, nails, screws, rods, pins, wire structures, sutures, anchor clips, staples, stents, bone grafts, biologics, cements, or the like.

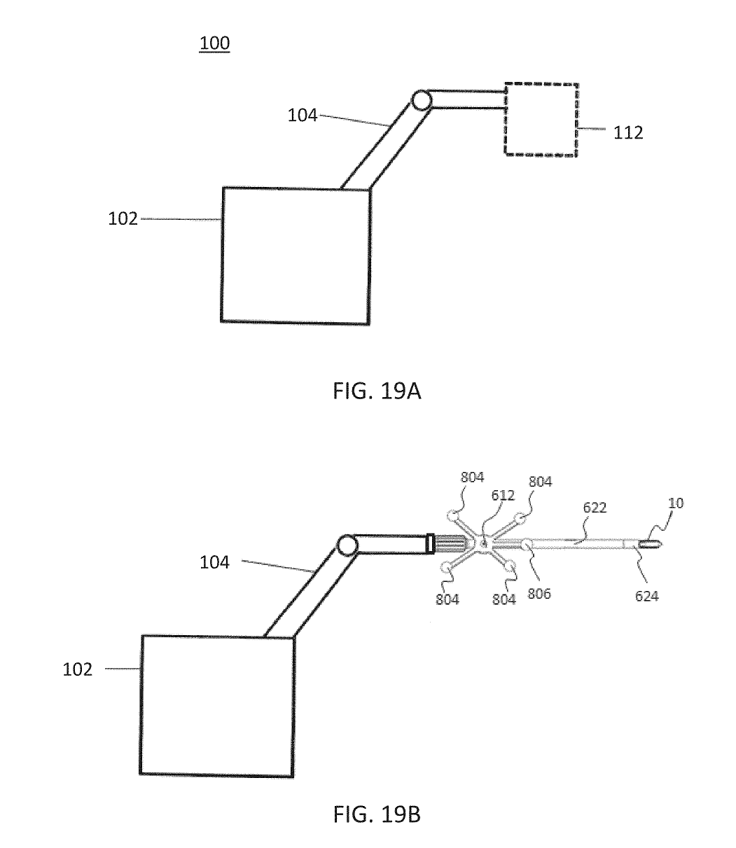

Turning now to FIG. 19A, it is envisioned that the robot end-effector 112 is interchangeable with other types of end-effectors 112. Moreover, it is contemplated that each end-effector 112 may be able to perform one or more functions based on a desired medical procedure. For example, the end-effector 112 having a guide tube 114 may be used for guiding an instrument 608 as described herein. In addition, end-effector 112 may be replaced with a different or alternative end-effector 112 that controls a medical device, instrument, or implant, for example.

The alternative end-effector 112 may include one or more devices or instruments coupled to and controllable by the robot. By way of non-limiting example, the end-effector 112, as depicted in FIG. 19A, may comprise a retractor (for example, one or more retractors disclosed in U.S. Pat. Nos. 8,992,425 and 8,968,363) or one or more mechanisms for inserting or installing medical devices such as expandable intervertebral fusion devices (such as expandable implants exemplified in U.S. Pat. Nos. 8,845,734; 9,510,954; and 9,456,903), stand-alone intervertebral fusion devices (such as implants exemplified in U.S. Pat. Nos. 9,364,343 and 9,480,579), expandable corpectomy devices (such as corpectomy implants exemplified in U.S. Pat. Nos. 9,393,128 and 9,173,747), articulating spacers (such as implants exemplified in U.S. Pat. No. 9,259,327), facet prostheses (such as devices exemplified in U.S. Pat. No. 9,539,031), laminoplasty devices (such as devices exemplified in U.S. Pat. No. 9,486,253), spinous process spacers (such as implants exemplified in U.S. Pat. No. 9,592,082), inflatables, fasteners including polyaxial screws, uniplanar screws, pedicle screws, posted screws, and the like, bone fixation plates, rod constructs and revision devices (such as devices exemplified in U.S. Pat. No. 8,882,803), artificial and natural discs, motion preserving devices and implants, spinal cord stimulators (such as devices exemplified in U.S. Pat. No. 9,440,076), and other medical devices. The end-effector 112 may include one or instruments directly or indirectly coupled to the robot for providing bone cement, bone grafts, living cells, pharmaceuticals, or other deliverable to a medical target. The end-effector 112 may also include one or more instruments designed for performing a discectomy, kyphoplasty, vertebrostenting, dilation, or other medical procedure.

The end-effector itself and/or the implant, device, or instrument may include one or more markers 118 such that the location and position of the markers 118 may be identified in three-dimensions. It is contemplated that the markers 118 may include active or passive markers 118, as described herein, that may be directly or indirectly visible to the cameras 200. Thus, one or more markers 118 located on an implant 10, for example, may provide for tracking of the implant 10 before, during, and after implantation.

As shown in FIG. 19B, the end-effector 112 may include an instrument 608 or portion thereof that is coupled to the robot arm 104 (for example, the instrument 608 may be coupled to the robot arm 104 by the coupling mechanism shown in FIGS. 9A-9C) and is controllable by the robot system 100. Thus, in the embodiment shown in FIG. 19B, the robot system 100 is able to insert implant 10 into a patient and expand or contract the expandable implant 10. Accordingly, the robot system 100 may be configured to assist a doctor or to operate partially or completely independently thereof. Thus, it is envisioned that the robot system 100 may be capable of controlling each alternative end-effector 112 for its specified function or medical procedure.

Although the robot and associated systems described herein are generally described with reference to spine applications, it is also contemplated that the robot system may be configured for use in other medical applications, including but not limited to, medical imaging, surgeries in trauma or other orthopedic applications (such as the placement of intramedullary nails, plates, and the like), cranial, neuro, cardiothoracic, vascular, colorectal, oncological, dental, and other medical operations and procedures.

During robotic spine (or other) procedures, a Dynamic Reference Base (DRB) may thus be affixed to the patient (e.g., to a bone of the patient), and used to track the patient anatomy. Since the patient is breathing, a position of the DRB (which is attached to the patient's body) may oscillate. Once a medical tool is robotically moved to a target trajectory and locked into position, patient movement (e.g., due to breathing) may cause deviation from the target trajectory even through the end-effector (e.g., medical tool) is locked in place. This deviation/shift (if unnoticed and unaccounted for) may thus reduce accuracy of the system and/or medical procedure.

Some Image Guided Surgery (IGS) systems include instruments and/or probes to be used in conjunction with a monitor display/interface. The 3D position and pose (also referred to as orientation) of the probe can be tracked by the system and can be displayed on the monitor with respect to an image of the patient. In some embodiments, it is desirable to indicate and store a specific location on a patient's image that may be subcutaneous and inaccessible without making an incision or burr hole. This location can be used as a target point of interest (e.g., a surgical plan) for guidance of different navigated instruments used through a procedure. The present disclosure describes methods to indicate and confirm a subcutaneous point of interest on the patient's image using the system's instrumentation. In some embodiments, the present disclosure describes mechanisms to indicate and confirm a desired position in 3D space beneath or within a patient without touching or directly interfacing with a monitor or display. In additional or alternative embodiments, the present disclosure describes image manipulation (e.g., zoom or rotation of the image) without touching or directly interfacing with a monitor or display.

Movement of a navigated probe relative to a 3D anatomical volume (e.g., a patient) can be monitored by a tracking system (also referred to as a positioning system). The navigated probe can be a pointer tool with an array of three or more tracked optical markers similar to the probe 608A in FIG. 13C. Examples of the navigated probe can include probes similar to probes currently used in commercially available navigation systems such as Medtronic StealthStation or BrainLAB VectorVision. Using a camera bar (e.g., camera 200), a tracking system can automatically read the 3D coordinates of the optical marker array 804 and calculate the location of the tip 624 of the probe 608A and a pose of a shaft 622 of the probe 608A. After registration of the camera and image coordinate system, the system can display in real-time a representation of the probe 608A in its tracked location and pose on the medical image volume. The system can further reformat the image volume so that the image is transformed to the local coordinate system of the probe 608A. This transformation of coordinates can allow standard 3-plane visualization of an anatomy on perpendicular slices through an image volume that are simultaneously coincident with the probe in all 3 planes.

Some probes can be used to find a spatial location of lesions, abnormalities, or other structures during medical procedures. For example, a surgeon might hold such a probe in view of tracking cameras and move it around to different locations outside or penetrating the patient while watching the changing medical image display. But, once a location is found it can be difficult to convey to the navigation system that this location needs to be stored in memory. A surgeon may call out to the technician that they should mark the location and the technician will manually store the location through software using mouse or keyboard entry. But, for a user to simultaneously hold the probe and click the mouse to store its position is a difficult maneuver to achieve without error and may require a sterile mouse. Furthermore, to explore locations that are internal to the patient, if the user does not want to physically penetrate the patient with the probe, extrapolation of the tip must be applied. With extrapolation applied, the system shows the location in line with the shaft but offset by some constant value. Some navigation systems allow software to extrapolate a probe's tip but require intervention by a user (typically a technician) to activate the feature in software and only a fixed extrapolation offset distance can be applied without requiring software adjustment. The present disclosure includes methods and mechanisms that allow a user to extrapolate the probe using a range of desired values and can mark locations without requiring assistance.

Before navigation begins, the user can indicate in software a distance to be used as a starting extrapolated offset to the probe. The probe can be moved free-hand to contact a desired point on a patient's skin. The user can adjust the angle of the probe to give a desired trajectory. A crosshair or other symbol, at a fixed distance along the probe's trajectory, may be displayed and used to indicate the point of interest on the patient's image. The crosshair is projected beyond the physical tip of the probe, such that the point of interest may be beneath the patient's skin.

When a point and trajectory are found and the user would like to store this position in the system's memory, the system can allow for several mechanisms to convey a signal or trigger to store the current position to the system. In some embodiments rocking the probe back and forth in excess of some minimum threshold angle several times (within a time window) followed by a delay period can be detected as a triggering event. For example, while keeping the tip of the probe fixed on the patient's skin, the user could rock the probe back and forth 3 times by at least 45 degrees within 2 seconds. Any deviation from this sequence could abort recording a point and would be perceived as just natural movement of the probe. In additional or alternative examples, the system can take this pivoting action as an indicator to store the trajectory and trigger a countdown, potentially displaying the countdown as a clock on the screen. After the countdown period, for example 3 seconds, a snapshot of the position of the probe can be stored. Such a method can allow the user to perform the action to trigger the countdown, then return the probe to the desired location and wait for the system to record the position.

In additional or alternative embodiments, it may be less tedious and error prone for the user to rotate the probe about the axis of its shaft a predetermined number of times in excess of some minimum threshold angle. Since rotation about the shaft axis changes neither the tip position nor the probe trajectory, this movement may be less likely to cause the system to record an undesired trajectory. Furthermore, no countdown period may be used, which may reduce the time for completing the process. For example, the system may detect the probe being rotated by at least 45 degrees 2 times within 2 seconds. An additional factor that could be considered for avoiding false positives is that the maneuver to trigger recording the point may be limited to movements that do not deviate from the recorded trajectory by more than a predetermined minimum angle (e.g., 5 degrees).

In additional or alternative embodiments, the system can detect the user pressing a foot pedal, chin switch, or another independent actuator as a trigger and record a trajectory of a probe in response to detecting the trigger. To avoid false positives, the system could wait, for example, until two or more sequential triggers are sensed within a predetermined time (e.g., 1 second) before recording the position. In some embodiments, the system could “undo” the last sensed movement within 1 second before recording the position. In additional or alternative embodiments, the system could “undo” the last trajectory if two or more sequential triggers are sensed, also providing visual confirmation on a display screen as a dialog, requesting an additional triggering to indicate that a trajectory should be removed from memory.

In additional or alternative embodiments, a system can record a trajectory in response to detecting a change in a number or size of confirmation markers on the probe that could be obstructed or unobstructed intentionally by a user. With a 4-marker probe, the marker that is obstructed then unobstructed could be one of the markers of the array since the probe position can be recorded with either 3 or 4 markers. In some examples, a probe can include an additional marker located at an easily accessed point such as the probe shaft. As with the triggering methods described above, this method could be limited such that the system detects a trigger in response to the marker being obstructed/unobstructed more than once within a certain time period to avoid false positives that may occur if the marker were accidentally obstructed during normal movement of the probe.

Optical tracking systems that use reflective markers can search for areas of contrast on 2D camera views that might represent the presence of reflective markers. Part of the recognition process can include assessing these contrasting regions or blobs for circularity and size since the reflective markers are spherical and have a known diameter. It is possible to use this recognition process in a slightly different way to cause a trigger event. For example, if a stripe of reflective paint was present on a rectangular flag attached to the shaft of the probe, the system may recognize this stripe as an elongated rectangular blob when evaluating contrasting areas, discarding it as a possible reflective marker. In response to the user placing their thumb or finger in the middle of this rectangle, the system may recognize two squares. The transition of the blob from a rectangular strip to a pair of squares with known separation could be a trigger/indicator to the system to record the probe trajectory. If the stripe were sized properly, a finger obstructing the central region could cause the system to visualize two small blobs that are roughly the size of optical markers. In this example, the tracking system may discern no marker (just an elongated blob) when the user is not obstructing the strip, but recognize 2 markers separated by a known distance and in a known relative position to the array markers when the user blocks the center section of the reflective stripe.

In additional or alternative embodiments, the user can be interested in defining a trajectory in which the desired tip does not correspond to the physical tip of the probe. In such cases, an offset can be applied through software to extrapolate the tip to a new virtual location that is still in line with the probe's shaft but is distal to the probe's physical tip. Using such a method allows the surgeon to keep the tip of the probe resting on the patient's skin while adjusting the angle of the probe and sliding along the skin to find the desired trajectory with more stability than may be possible while holding the probe hovering freely. However, for a user to change the amount of extrapolation to find a point deeper or shallower than the current extrapolation while continuing to hold the probe against the patient's skin can be difficult without assistance.

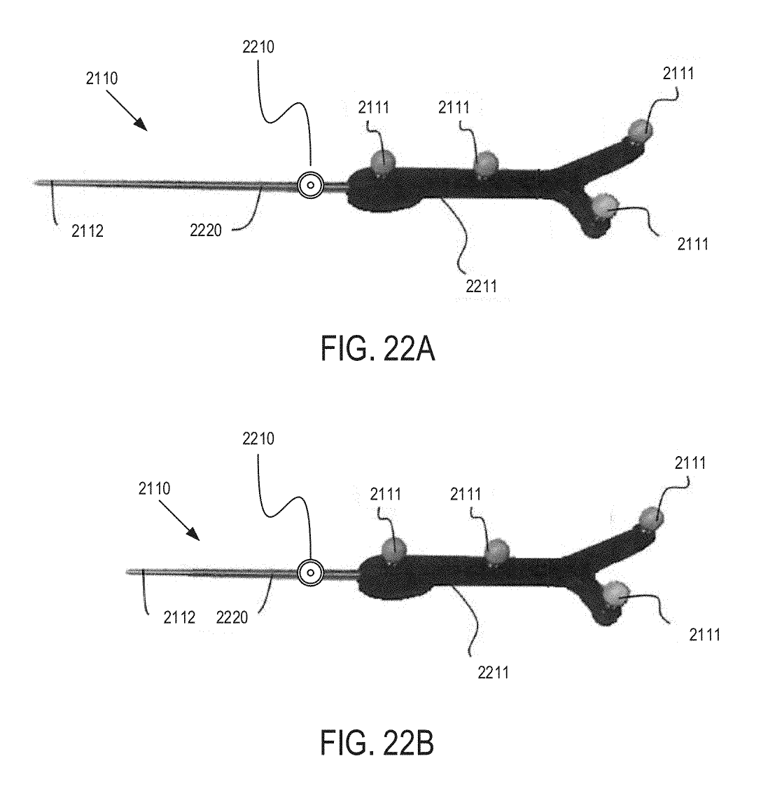

As depicted in FIGS. 22A-B and further described below, a probe 2110 with a telescoping shaft 2220 could allow the user to adjust the spacing between the tip 2112 and the tracked array of the probe 2110 dynamically. The lower telescoping portion could be held against the patient's skin for stability. The upper telescoping portion can have the navigation tracker 2211 (e.g., passive maker array) attached to it. With the virtual extrapolated probe tip being at a fixed distance from the navigation tracker 2211, expanding or collapsing the telescoping shaft 2220 of the probe 2110 would change the extrapolated probe tip position relative to the physical probe tip 2112 position. The mechanism for causing the probe 2110 to telescope in or out (e.g., collapse or expand) could be a simple friction fit that the user pushes together or pulls apart. Alternatively, a button could first be actuated to unlock the telescoping mechanism then to relock it after collapsing or expanding. Alternatively, a first button on the probe 2110 could allow the probe 2110 to expand and a second button to collapse. Such a method could be especially useful, for example, in identifying an anatomical landmark deep inside the brain. The user could guess an initial extrapolation distance, rest the probe tip 2112 on the patients' skull, and maneuver the probe 2110 around until a displayed location is close to the desired location. If the landmark is deeper into the brain than the current extrapolation, the user could collapse the telescopable probe 2110; if the landmark is shallower than the current extrapolation, the user could the expand the probe 2110 while keeping the physical tip 2112 of the probe 2110 resting on the patient's skull for stability. The point and trajectory could be confirmed by any of the methods described above such as rotating the probe 2110 about its axis, pressing the food pedal, or occluding/un-occluding a maker without requiring any direct interrogation via mouse/keyboard/touchscreen with the software.

In additional or alternative embodiments, an alternative to the telescoping probe design that also offers additional view changing functionality is a single-piece probe with another method of adjusting the extrapolation distance through sequences of movements. One embodiment of such a method would be to enter a view-change mode through some sequence of movements, for example, repeatedly obstructing/unobstructing a trigger marker within a time frame or repeatedly pivoting the probe. Once in view-change mode, different actions for the probe could correspond to different view change behaviors. For example, rocking the probe left to right could move the visual display move up or down along the trajectory (change the extrapolation distance). A rapid left-to-right movement followed by a slow right-to-left movement could indicate that extrapolation should increase while rapid right-to-left followed by slow left-to-right could mean that extrapolation should decrease. Rotating the probe while in view-change mode could pin the visual display about the axis of the probe. Moving the probe toward or way from the camera could zoom in or out.

The present disclosure describes mechanisms to allow the probe to navigate to a desired point, to manipulate the images, and with a gesture of the probe or an external trigger to record a location. The surgeon does not need to directly interface with software through a mouse/keypad or touch screen to perform these tasks. Since the user does not need to touch the display or computer, navigation can be done at the patient's side and not at the computer screen. Furthermore, planning a trajectory using a probe instead of by drawing the plan on the screen may provide the additional advantage that the surgeon can create the surgical plan while taking into consideration both the point of interest and the path that instrument will take to reach the point of interest. The surgical plan that the surgeon creates can then be relayed to a surgical robot, which can then move to this trajectory and hold the trajectory while the surgeon places implants or performs other surgical procedures.

Elements of robotic systems discussed above may be used to indicate and confirm a desired position on an image of 3D space within an anatomical body or to manipulate the image without touching or directly interfacing with a monitor or display. By using navigated tools without interfacing with a monitor, surgeons can complete free-hand planning of instrument and implant locations without leaving the patient's side. The present disclosure further allows surgeons to manipulate navigation screen properties, including extrapolation distance, rotation angle, and zoom using only the navigation probe. As a result, surgeons can have a more user-friendly system and more flexibility when planning robotic or navigated surgical cases. The system can offer surgeons greater flexibility because the motions performed in moving the probe to determine a location within the patient may mimic the motions of a surgery to be performed at the location in a body. The system can also make planning implants insertion at the location with correct offsets easier and better leverage the perspective of the surgeon.

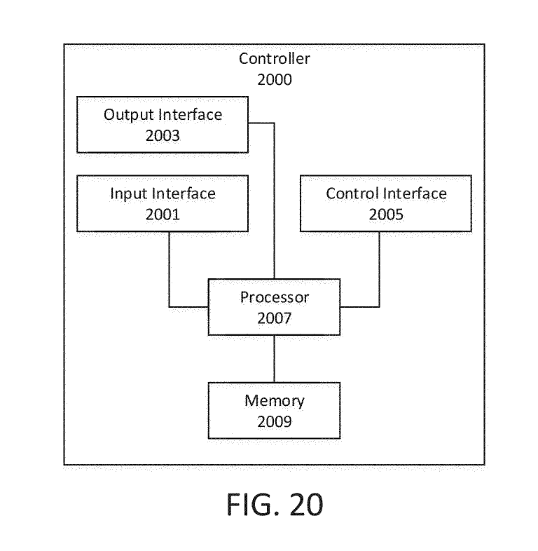

FIG. 20 is a block diagram illustrating elements of a controller 2000 (e.g., implemented within computer 408) for a robotic image-guided surgical system. As shown, controller 2000 may include processor circuit 2007 (also referred to as a processor) coupled with input interface circuit 2001 (also referred to as an input interface), output interface circuit 2003 (also referred to as an output interface), control interface circuit 2005 (also referred to as a control interface), and memory circuit 2009 (also referred to as a memory). The memory circuit 2009 may include computer readable program code that when executed by the processor circuit 2007 causes the processor circuit to perform operations according to embodiments disclosed herein. According to other embodiments, processor circuit 2007 may be defined to include memory so that a separate memory circuit is not required.

As discussed herein, operations of image-guided surgical system controller 2000 (e.g., a robotic navigation system controller) may be performed by processor 2007, input interface 2001, output interface 2003, and/or control interface 2005. For example, processor 2007 may receive user input through input interface 2001, and such user input may include user input received through a keyboard, mouse, touch sensitive display, foot pedal 544, tablet 546, etc. Processor 2007 may also receive position sensor input from tracking system 532 and/or cameras 200 through input interface 2001. Processor 2007 may provide output through output interface 2003, and such output may include information to render graphic/visual information on display 304 and/or audio output to be provided through speaker 536. Processor 2007 may provide robotic control information through control interface 2005 to motion control subsystem 506, and the robotic control information may be used to control operation of robot arm 104 or other robotic actuators. Processor 2007 may also receive feedback information through control interface 2005.

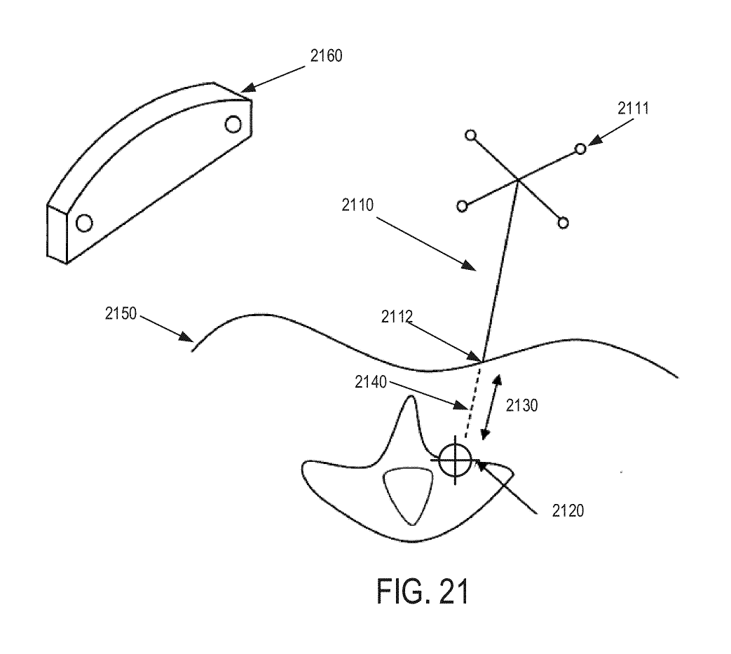

FIG. 21 depicts an example of an image-guided surgical system used to indicate and confirm a desired location within an anatomical body 2150. In this example, the image-guided surgical system allows the positioning of a virtual crosshair 2120 at a location on an anatomical image in response to movement of a probe 2110. The image-guided surgical system includes sensors 2160, which can capture movement of a probe 2110 with a tip 2112 in relation to the body 2150. The sensors 2160 can include cameras 200, the probe 2110 can include navigated instrument 608 as discussed above with respect to FIG. 13C, and the body 2150 can include patient 210. The image-guided surgical system can determine a trajectory 2140 (e.g., a location and a direction) based on a pose of the probe 2110 and can determine a position of a virtual crosshair 2120 based on an offset 2130 from the tip 2112 along the trajectory 2140. The image-guided surgical system can further store a location of the virtual crosshair 2120 in response to detecting a trigger based on information regarding the probe 2110.

In some embodiments, the probe 2110 can be a physical device that is positioned adjacent to the body 2150, which can include a human body. The image-guided surgical system can use 3D anatomical image information to generate an image of the body 2150 that can include a location of the virtual crosshair 2120. An image based on the anatomical image information including the virtual crosshair 2120 can be displayed on a monitor and updated such that moving the probe 2110 adjacent to the body 2150 can cause a virtual implementation of the probe 2110, trajectory 2140, and/or the virtual crosshair 2120 to move on the image displayed. The system can track the probe 2110, and based on detecting a trigger, store a location identified using the probe 2110. The user can perform the triggering event in several ways without directly or manually interfacing with the system. In some embodiments, the position of the tip 2212 of the probe 2110 can be used to detect a trigger. In additional or alternative embodiments, the trigger can be detected based on detecting a rotation of the probe 2110. In additional or alternative embodiments, the processor 2007 can, on a first press of a button (e.g., a foot pedal), set the trajectory 2140 and determine a placement for the virtual crosshair 2120 at a default offset along the trajectory 2140. Holding the button can allow for further adjustment of the offset 2130 along the trajectory 2140 and the release of the button can trigger the processor 2007 to store the location in memory. In additional or alternative embodiments, the processor 2007 can detect a trigger based on a change to the probe 2110. In some examples, the processor 2007 can detect a trigger based on one of the tracker markers 2111 being fully or partially obstructed. In additional or alternative examples, the probe 2111 can include a telescoping shaft and the processor 2007 can detect a trigger based on the length of the probe 2110 being adjusted.

For purposes of illustration, FIG. 21 mixes physical elements (directly visible to a user) and virtual elements (that are only visible on a display). In particular, a surface of body 2150 and/or the physical probe 2110 may be visible to the user and sensor 2160, but internal portions of the body (e.g., the virtual crosshair 2120 and trajectory 2140) are not directly visible. Instead, the surgical navigation system may render a two dimensional image on a display, with the two dimensional image including internal portions of body 2150, virtual crosshair 2120, the surface of body 2150, and probe 2110.

Examples of a telescoping version of the probe 2110 are depicted in FIGS. 22A-B. The same probe 2110 is depicted in both FIG. 22A and 22B. The probe includes a thumb wheel 2210 that may be turned to adjust a length of the probe 2110. By turning the thumb wheel 2210 in FIG. 22A clockwise the telescoping shaft 2220 retracts upon itself and shortens to the length of the telescoping shaft 2220 in FIG. 22B. In some embodiments, the thumb wheel 2210 can include a tracking marker 2111 on a portion of the wheel such that rotating the thumb wheel 2210 can switch between states of revealing and covering the tracking marker 2111. The processor 2007 can detect the trigger based on changes between the states of the thumb wheel 2210. In additional or alternative embodiments, the probe 2110 can include a tracking marker 2111 on the telescoping shaft of the probe 2110 such that the processor 2007 can detect changes in the length of the probe 2110. The processor 2007 can detect a trigger based on a change in the length of the probe 2110 or based on the length being above or below a threshold length. In additional or alternative embodiments, the processor 2007 can be provided with positional data for the tracking markers 2111 and a default length of the tip 2112 from the tracking markers 2111. The processor 2007 can detect a trigger based on determining that the positional data for the tracking markers 2111 and the default length would indicate a virtual representation of the tip is within the body. For example, if a user shortens a probe 2110 using the thumb wheel 2210 while maintaining the position of the tip 2112 on the surface of the body, the processor 2007 will calculate the position of the tip 2112 to be within the anatomical body 2150 and can detect this change as a trigger.

Although FIGS. 22A-B depict a telescoping version of the probe 2110 with a thumb wheel 2210, other mechanisms can be used to adjust the length of the probe 2110. Additional elements of the probe 2110 (e.g., the tracking marker 2111) are explained with more detail in regards to the medical instrument in FIGS. 13A-C above.

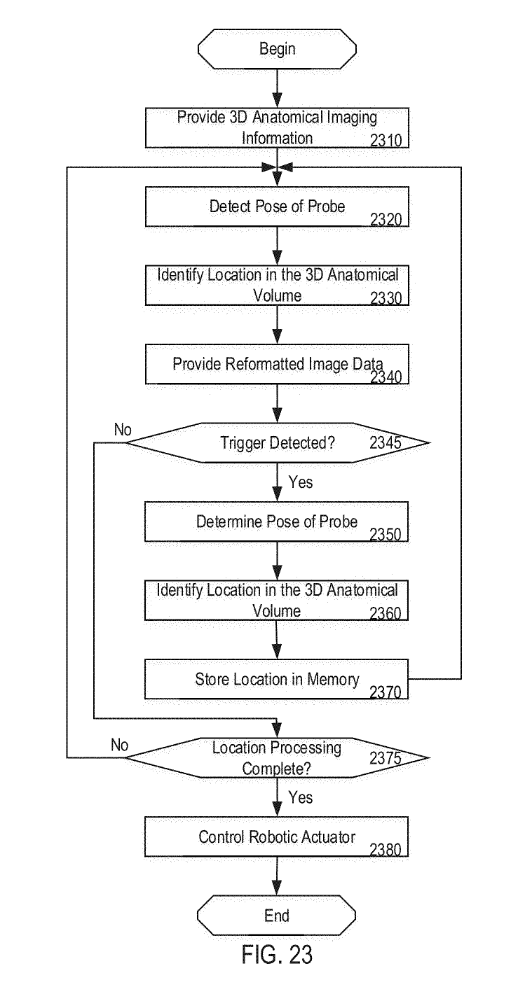

Operations of an image-guided surgical system (e.g., a robotic navigation system that includes a robotic actuator configured to position a probe on a body) will now be discussed with reference to the flow chart of FIG. 23 according to some embodiments. For example, modules may be stored in memory 2009 of FIG. 20, and these modules may provide instructions so that when the instructions of a module are executed by processor 2007, processor 2007 performs respective operations of the flow chart of FIG. 23.

FIG. 23 depicts an example of operations performed by an image-guided surgical system using imaging information for a 3D anatomical volume to identify and store a location in the 3D anatomical volume based on the pose of the probe 2110 and based on imaging information.

At block 2310, processor 2007 may provide 3D anatomical imaging information for a 3D anatomical volume (e.g., the body 2150). The 3D anatomical imaging information can be provided, for example, based on a previously performed CT scan, MRI, or fluoroscopic images. At block 2320, processor 2007 may detect a pose of the probe 2110. The pose of the probe 2110 can include the position and/or the orientation of the probe 2110. In some embodiments, processor 2007 may detect the pose based on information received from a tracking system (e.g., including cameras 200). For example, the probe 2110 may include one or more spaced apart tracking markers 2111, and the tracking system may detect the pose of the probe 2110 by detecting the tracking markers 2111. The processor 2007 can receive position data or orientation data from the tracking system via input interface 2001.

At block 2330, processor 2007 may identify a location in the 3D anatomical volume. The location can be identified based on a pose of the probe and the imaging information. In some embodiments, processor 2007 can identify the location by determining the trajectory 2140 that includes the location and a direction. In additional or alternative embodiments, the location can be a location of the probe 2110 or the virtual crosshair 2120.

At block 2340, processor 2007 may provide reformatted image data via output interface 2003. In some embodiments, the processor 2007 provides the reformatted image data to be rendered on a display (e.g., display 110) based on the 3D anatomical imaging information and the pose of the probe. The reformatted image data can identify the location of the virtual crosshair 2120 in an image based on the 3D anatomical imaging information.

In additional or alternative embodiments, processor 2007 may provide new image data based on capturing new image information of the 3D anatomical volume. The new image data can include the location of the virtual crosshair 2120 such that an image (e.g., a 2D slice) of the 3D anatomical volume can be generated that includes the virtual crosshair 2120.

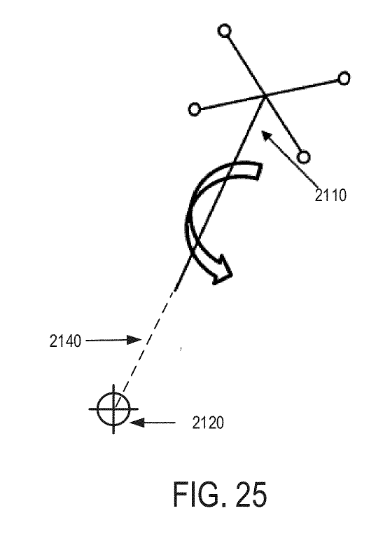

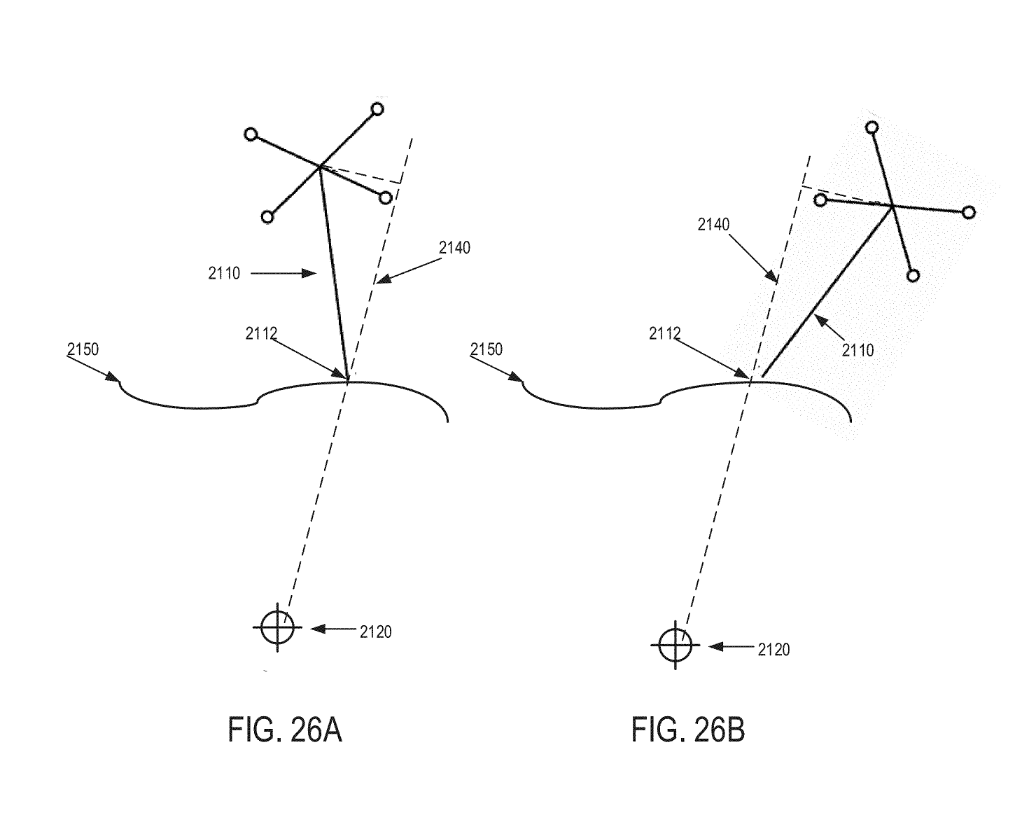

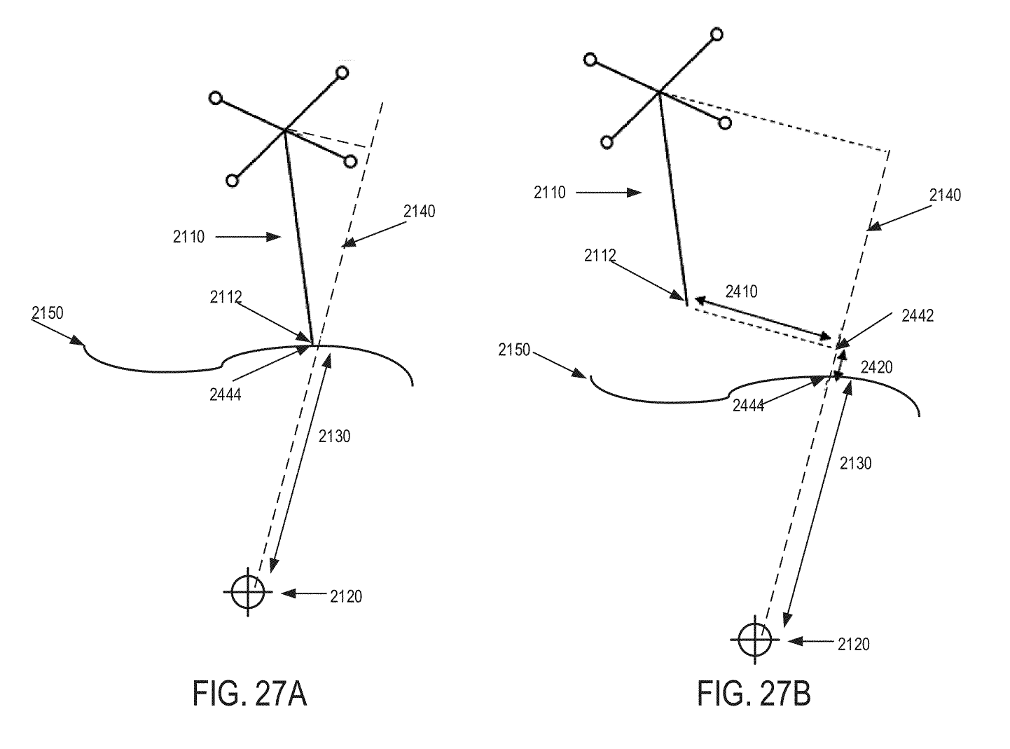

If processor 2007 has not detected a trigger, then at block 2345 the processor 2007 may proceed to block 2375. If the processor 2007 has detected a trigger, then at block 2345 the processor 2007 may proceed to block 2350. In some embodiments, processor 2007 can receive position data for the probe 2110 indicating the probe has moved in a predefined way so as to indicate a desire to store the location in the 3D anatomical volume. Such triggers are discussed in greater detail with respect to FIGS. 25, 26A-B, and 27A-B.

In some embodiments, detecting the trigger can include detecting a rotation of the probe about a longitudinal axis of the probe. In additional embodiments, detecting the trigger can include detecting rotation in a first direction and then rotation in a second direction, a number of changes in direction of rotation in a defined period of time, or a rotation of at least a predetermined number of degrees. Such triggers are discussed in greater detail with respect to FIG. 25.

In additional or alternative embodiments, detecting the trigger can include detecting a change in an angular orientation of a longitudinal axis of the probe. In additional embodiments, detecting the trigger can include detecting a change of angular orientation in a first direction and then a change of angular orientation in a second direction, a number of changes in angular direction in a defined period of time, and/or a change in angular orientation of at least a predetermined number of degrees. Such triggers are discussed in greater detail with respect to FIGS. 28A-B.

In additional or alternative embodiments, the probe 2110 can include one or more tracking markers 2111 and detecting the trigger can include detecting an obstruction of at least one of the tracking markers 2111. The probe can include a shaft that defines a longitudinal axis with more than one tracking markers 2111 coupled to the shaft. Detecting the pose can include detecting the pose based on information received from the tracking system regarding the tracking markers 2111 and identifying the location can include identifying the location at a predefined distance from an end of the shaft in a direction of the longitudinal axis.

Blocks 2350 and 2360 are similar to blocks 2320 and 2330 respectively. At block 2350, processor 2007 determines a pose of the probe 2110. At block 2360, processor 2007 can identify a location in the 3D anatomical volume. In some embodiments, in blocks 2320 and 2330 the processor 2007 can detect a first pose and identify a first location based on first reformatted image data. In blocks 2350 and 2360, the processor 2007 can detect a second pose and identify a second location based on second reformatted image data. In additional or alternative embodiments, a timer can be set at block 2345 and the processor 2007 can determine the pose and identify the location in response to expiration of the timer.

At block 2370, processor 2007 stores the location in memory 2009. In some embodiments, identifying includes identifying the location and a direction toward the location based on detecting the pose of the probe 2110 and based on detecting the trigger. Storing can include storing the location and the direction in the memory 2009 based on detecting the pose of the probe 2110 and based on detecting the trigger.

After storing the location in memory, processor 2007 can repeat blocks 2320, 2330, and 2340. Repeating blocks 2320 and 2330 may cause the processor 2007 to detect one or more additional poses and identify one or more additional locations. In repeating block 2340, processor 2007 may provide second reformatted image data to be rendered on the display based on the 3D anatomical imaging information and based on the second pose of the probe 2110. The second reformatted image data may identify the second location in a second image that is different than the first location in the first image.

If a new trigger is detected then processor 2007 can repeat blocks 2350, 2360, and 2370 to store a new location. A plurality of locations may thus be identified and stored according to some embodiments. If no trigger is detected and the location processing is complete then in block 2375 processor 2007 can proceed to block 2380. Having stored one or more locations as discussed above, reformatted image data of block 2340 may be used to simultaneously display a current location identified based on a current pose of the probe and one or more of the stored locations.

At block 2380 processor 2007 may control a robotic actuator. In some embodiments, processor 2007 can control a robotic actuator via control interface 2005 to position an end-effector at the location stored in memory. For example, the processor 2007 can control a robotic actuator to position an end-effector such that a surgical operation can be performed using the end-effector to place a physical implant at a position in a body based on the location. In additional or alternative embodiments, processor 2007 can control the robotic actuator to perform the surgical operation and place a physical implant in the body based on the information stored in memory 2009.

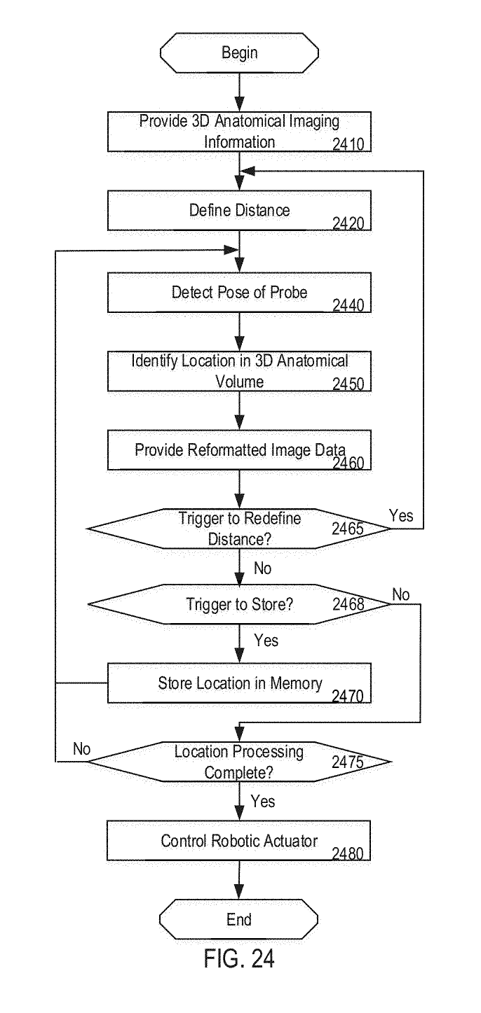

FIG. 24 depicts an example of operations performed by an image-guided surgical system using imaging information for a 3D anatomical volume to manipulate an image of a 3D anatomical volume based on manipulation of the probe 2110.

At block 2410, processor 2007 may provide 3D anatomical imaging information for a 3D anatomical volume (e.g., the body 2150). The 3D anatomical imaging information may be provided, for example, based on a previously performed CT scan, MM, or fluoroscopic imaged. At block 2420 the processor 2007 can define a first distance (also referred to as an offset) from an end of the probe 2110.

At block 2440, processor 2007 may detect a first pose of a probe based on information received from the tracking system. For example, the probe 2110 may include one or more spaced apart tracking markers 2111 and the tracking system may detect the pose of the probe 2110 by detecting the tracking markers 2111. The processor 2007 can receive position data or orientation data from the tracking system (e.g., including cameras 200) via input interface 2001.

At block 2450, processor 2007 may identify a first location in the 3D anatomical volume based on the first pose of the probe, the imaging information, and the first distance from an end of the probe. In some embodiments, processor 2007 identifies the trajectory 2140 that includes the location and a direction. The location can be identified based on pose of the probe and the imaging information.

At block 2460, processor 2007 can provide reformatted image data via output interface 2003. In some embodiments, the processor 2007 provides the reformatted image data to be rendered on a display 110 based on the 3D anatomical imaging information and the pose of the probe. The reformatted image data can identify the location of the virtual crosshair 2120 in an image based on the 3D anatomical imaging information.

If processor 2007 detects a trigger to redefine the distance based on information received from the position system regarding the probe after identifying the first location, then at block 2465, the processor returns to block 2420 and repeats blocks 2420, 2440, 2450, and 2460. In some embodiments, processor 2007 provides second reformatted image data when repeating block 2460. The second reformatted image data may be different than the first reformatted image data provided previously in block 2460. For example, the trajectory 2140 including the location and/or the direction may have changed.

If the processor 2007 detects a trigger to store the location then at block 2468 proceeds to block 2470, stores the location in the memory 2009, and repeats blocks 2440, 2450, and 2460. If the processor 2007 detects no triggering event, but determines the location processing is not complete then at block 2475, the processor returns to block 2440 and repeats blocks 2440, 2450, and 2460. If the processor 2007 determines the location processing is complete, then at block 2475 the processor proceeds to block 2480.

In some embodiments, detecting a trigger at block 2465 or block 2468 can include detecting a rotation of the probe about a longitudinal axis of the probe. In additional embodiments, detecting the trigger can include detecting rotation in a first direction and then rotation in a second direction, a number of changes in direction of rotation in a defined period of time, or a rotation of at least a predetermined number of degrees. Such a trigger is discussed further in respect to FIG. 25.

In additional or alternative embodiments, detecting a trigger can include detecting a change in an angular orientation of a longitudinal axis of the probe. In additional embodiments, detecting a trigger can include detecting a change of angular orientation in a first direction and then a change of angular orientation in a second direction, a number of changes in angular direction in a defined period of time, or a change in angular orientation of at least a predetermined number of degrees. Such a trigger is discussed further in respect to FIGS. 26A-B.

In additional or alternative embodiments, the probe 2110 can include one or more tracking markers 2111, and detecting a trigger can include detecting an obstruction of at least one of the tracking markers 2111. The probe 2110 can include a shaft that defines a longitudinal axis with more than one tracking marker 2111 coupled to the shaft. Detecting the pose can include detecting the pose based on information received from the tracking system regarding the tracking markers 2111 and identifying the location can include identifying the location at a predefined distance from an end of the shaft in a direction of the longitudinal axis.

In additional or alternative embodiments, detecting a trigger can include detecting a change in a position of the tip 2112 of the probe 2110. Such a trigger is discussed further in respect to FIGS. 27A-B.

At block 2480 processor 2007 may control a robotic actuator. In some embodiments, processor 2007 can control a robotic actuator via control interface 2005 to position an end-effector at the location stored in memory. For example, the processor 2007 can control a robotic actuator to position an end-effector such that a surgical operation can be performed using the end-effector to place a physical implant at a position in a body based on the location. In additional or alternative embodiments, processor 2007 can control the robotic actuator to perform the surgical operation and place a physical implant in the body based on the information stored in memory 2009.

FIG. 25 depicts an example of how a rotation of the probe 2110 about a longitudinal axis of the probe 2110 can be detected as a trigger by the processor 2007. In some embodiments, processor 2007 can store a location in response to the trigger, manipulate a 3D anatomical image, or adjust an offset for the virtual crosshair 2120 while maintaining a direction/pose of the trajectory 2140. In some examples, processor 2007 can zoom in on a portion of the image depicting the trajectory 2140 and virtual crosshair 2120 in response to detecting a clockwise rotation of the probe 2110 about the longitudinal axis of the probe 2110. The processor 2007 can zoom out on the image in response to detecting a counter-clockwise rotation of the probe 2110 about the longitudinal axis of the probe 2110. In additional or alternative embodiments, the processor 2007 may detect the rotation of the probe 2110 and determine to store a location (e.g., of the virtual crosshair 2120) in memory as in block 2468 in FIG. 24. This can allow the user to store the location while holding the probe 2110 in position.

In additional or alternative embodiments, the speed of the movement or rotation of the probe 2110 can be determined by the probe 2110 and used to determine a type of trigger being detected. For example, a quick rotation can be detected as a trigger to store the location and a slower rotation can be detected as a trigger to manipulate the image (e.g., zoom in or zoom out).

In some embodiments, processor 2007 can implement a ratcheting system such that adjustments to the position of the virtual crosshair 2120 are only made in response to rotation of the probe 2110 in one direction. For example, the processor 2007 may only adjust the offset of the virtual crosshair 2120 in response to detecting clockwise rotation of the probe 2110 allowing a user to adjust the virtual crosshair 2120 farther in a single direction along the trajectory 2140 while maintaining a small working envelope or with greater precision. In additional or alternative embodiments, the probe 2110 may be rendered on the display as a virtual tap or other virtual surgical instrument or implant corresponding most closely to the implant-instrument combination that may be used in a subsequent surgery.

FIGS. 26A-B depict examples of how a change in the pose of the probe 2110 can be used to trigger storage of a location or manipulation of an image. In regards to FIG. 26A, the probe 2110 has been tilted such that a longitudinal axis of the probe 2110 is not aligned with the previously determined trajectory 2140. FIG. 26B depicts the probe 2110 tilted in another direction. The processor 2007 can detect movement between two different tilted positions and determine the movement is a trigger. In some embodiments, the processor 2007 determines the movement is a trigger based on the probe 2110 being moved back and forth (e.g., wiggled) between two tilted positions a predetermined number of times within a predetermined period of time. In some embodiments, the processor 2007 can store the trajectory 2140 in response to detecting the wiggle. In additional or alternative embodiments, the processor 2007 can manipulate the image in response to detecting the wiggle. In some examples, the processor 2007 can detect the probe 2110 being quickly tilted toward the sensor 2160 and subsequently tilted slowly away from the camera and determine to zoom in on the image. In additional or alternative examples, the processor 2007 can detect the probe 2110 being quickly tilted away from the sensor 2160 and subsequently tilted slowly toward the camera and determine to zoom out on the image.

FIGS. 27A-B depict examples of how a position of the tip 2112 of the probe 2110 can be used to trigger storage of a location or manipulation of an image. In regards to FIG. 27A, processor 2007 can use position data of the tip 2112 of the probe 2110 to determine an initial position 2444 of the tip 2112, which may be on the trajectory 2140. An offset 2130 of the virtual crosshair 2120 from the initial position 2444 can be set to a default offset that extends into the 3D anatomical volume (body 2150 in this example). FIG. 27B depicts the probe 2110 in another position relative to the body 2150. As probe 2110 moves, processor 2007 can determine a closest corresponding point 2442 along the trajectory to the tip 2112. Processor 2007 can further determine a distance 2420 between the closest corresponding point 2442 and the initial position 2444 of the tip 2112. In some embodiments, distance 2420 can be used to manipulate the image being displayed. For example, as distance 2420 increases the image may be zoomed out. In additional or alternative embodiments, processor 2007 can detect a trigger as occurring if a change in distance 2420 exceeds a threshold value within a predetermined period of time.

In additional or alternative embodiments, processor 2007 can determine the distance 2410 between the initial position 2444 of the tip 2112 and the current location of the tip 2112. The closest point 2442 may remain the same while the offset is adjusted based on changes in the distance 2410. In some embodiments, distance 2410 can be used to manipulate the image being displayed. For example, as distance 2410 increases the image may be zoomed out. In additional or alternative embodiments, processor 2007 can detect a trigger as occurring if a change in distance 2410 exceeds a threshold value within a predetermined period of time.

Any of the examples in FIGS. 25, 26A-B, and 27A-B can be combined for storing location data, manipulating the 3D anatomical image, and adjusting the placement of the virtual crosshair 2120 along the trajectory 2140 without interfacing with a monitor or display. In any of the embodiments, the ratio of user motion to adjustment amount can be scaled. The scaling may be informed by the position and pose of the navigated instrument. For example, the processor 2007 can change the scaling when using a rotational mode based on changing the pitch of the probe 2110 or a distance between a point on the probe 2110 and a point on the trajectory 2140.

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belong. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components, or functions but does not preclude the presence or addition of one or more other features, integers, elements, steps, components, functions, or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Although several embodiments of inventive concepts have been disclosed in the foregoing specification, it is understood that many modifications and other embodiments of inventive concepts will come to mind to which inventive concepts pertain, having the benefit of teachings presented in the foregoing description and associated drawings. It is thus understood that inventive concepts are not limited to the specific embodiments disclosed hereinabove, and that many modifications and other embodiments are intended to be included within the scope of the appended claims. It is further envisioned that features from one embodiment may be combined or used with the features from a different embodiment(s) described herein. Moreover, although specific terms are employed herein, as well as in the claims which follow, they are used only in a generic and descriptive sense, and not for the purposes of limiting the described inventive concepts, nor the claims which follow. The entire disclosure of each patent and patent publication cited herein is incorporated by reference herein in its entirety, as if each such patent or publication were individually incorporated by reference herein. Various features and/or potential advantages of inventive concepts are set forth in the following claims.

What is claimed is:

1. A method of operating an image-guided surgical system using imaging information for a 3-dimensional anatomical volume, the method comprising:
detecting a trigger based on information received from a tracking system regarding a probe;
detecting a pose of the probe based on the information received from the tracking system;
identifying a location in the 3-dimensional anatomical volume based on the pose of the probe and based on the imaging information; and
storing the location in memory based on detecting the trigger and detecting the pose of the probe.

2. The method of claim 1 further comprising:
providing reformatted image data to be rendered on a display based on the imaging information and based on the pose of the probe, the imaging information including 3-dimensional anatomical imaging information for the 3-dimensional anatomical volume, wherein the reformatted image data identifies the location in an image based on the 3-dimensional anatomical imaging information.

3. The method of claim 2, wherein the pose of the probe is a first pose of the probe, wherein the location is a first location, and wherein the reformatted image data is first reformatted image data, and wherein the image is a first image, the method further comprising:
after storing the location in memory and after providing the first reformatted image data, detecting a second pose of the probe based on information receiving from the tracking system;
identifying a second location in the 3-dimensional anatomical volume based on the pose of the probe and based on the imaging information; and
providing second reformatted image data to be rendered on the display based on the 3-dimensional anatomical imaging information and based on the second pose of the probe, wherein the second reformatted image data identifies the second location in a second image based on the imaging information.

4. The method of claim 1, wherein detecting the trigger comprises detecting a rotation of the probe about a longitudinal axis of the probe.

5. The method of claim 1, wherein detecting the trigger comprises detecting a change in an angular orientation of a longitudinal axis of the probe.

6. The method of claim 1, wherein the probe comprises a plurality of spaced apart tracking markers, wherein detecting the trigger comprises detecting obstruction of at least one of the tracking markers.

7. The method of claim 1, wherein the probe comprises a plurality of spaced apart tracking markers and an elongated reflective section, wherein detecting the trigger comprises determining a first portion of the elongated reflective section is obstructed by detecting a second portion and a third portion of the elongated reflective section as additional tracking markers.

8. The method of claim 1 further comprising:
setting a timer responsive to detecting the trigger;
wherein identifying the location comprises identifying the location based on the pose of the probe at expiration of the timer.

9. The method of claim 1, wherein the probe comprises a shaft defining a longitudinal axis and a plurality of tracking markers coupled with the shaft, wherein detecting the pose comprises detecting the pose based on information received from the tracking system regarding the tracking markers, and wherein identifying the location comprises identifying the location at a predefined distance from an end of the shaft in a direction of the longitudinal axis.

10. The method of claim 1, wherein identifying comprises identifying the location and a direction toward the location based on detecting the pose of the probe and based on detecting the trigger, and wherein storing comprises storing the location and the direction in memory based on detecting the pose of the probe and based on detecting the trigger.

11. The method of claim 10 further comprising:
controlling a robotic actuator to position an end-effector based on the location and the direction stored in memory.

12. A method of operating an image-guided surgical system using imaging information for a 3-dimensional anatomical volume, the method comprising:
defining a first distance from an end of a probe;
detecting a first pose of a probe based on information received from the tracking system;
identifying a first location in the 3-dimensional anatomical volume based on the first pose of the probe, based on the 3-dimensional imaging information, and based on the first distance from an end of the probe;
detecting a trigger based on information received from the tracking system regarding the probe after identifying the first location;
defining a second distance from the end of the probe responsive to detecting the trigger;
detecting a second pose of the probe based on information received from the tracking system; and
identifying a second location in the 3-dimensional anatomical volume based on the second pose of the probe, based on the imaging information, and based on the second distance from the end of the probe.

13. The method of claim 12 further comprising:
providing first reformatted image data to be rendered on a display based on the imaging information and based on the first pose of the probe, wherein the imaging information includes 3-dimensional anatomical imaging information for the 3-dimensional anatomical volume, wherein the first reformatted image data identifies the first location in a first image based on the 3-dimensional anatomical imaging information; and
providing second reformatted image data to be rendered on the display based on the 3-dimensional anatomical imaging information and based on the second pose of the probe, wherein the second reformatted image data identifies the second location in a second image based on the 3-dimensional anatomical imaging information.

14. The method of claim 12, wherein detecting the trigger comprises detecting a rotation of the probe about a longitudinal axis of the probe.

15. The method of claim 12, wherein detecting the trigger comprises detecting a change in an angular orientation of a longitudinal axis of the probe.

16. The method of claim 12, wherein the probe comprises a plurality of spaced apart tracking markers, wherein detecting the trigger comprises detecting obstruction of at least one of the tracking markers.

17. The method of claim 12, wherein the probe comprises a plurality of spaced apart tracking markers and an elongated reflective section, wherein detecting the trigger comprises determining a first portion of the elongated reflective section is obstructed by detecting a second portion and a third portion of the elongated reflective section as additional tracking markers.

18. The method of claim 12, wherein the probe comprises a shaft defining a longitudinal axis and a plurality of tracking markers coupled with the shaft, wherein detecting the first and second poses comprises detecting the first and second poses based on information received from the tracking system regarding the tracking markers, wherein identifying the first location comprises identifying the first location at the first distance from the end of the shaft in a direction of the longitudinal axis, and wherein identifying the second location comprises identifying the second location at the second distance from the end of the shaft in the direction of the longitudinal axis.

19. The method of claim 12 further comprising:
storing the second location in memory based on detecting the second pose of the probe.

20. An image-guided surgical system using 3-dimensional anatomical imaging information for a 3-dimensional anatomical volume, the image-guided surgical system comprising:
a processor; and
memory coupled with the processor, wherein the memory comprises instructions stored therein, and wherein the instructions are executable by the processor to cause the processor to:
detect a trigger based on information received from a tracking system regarding a probe;
detect a pose of the probe based on information received from a tracking system;
identify a location in the 3-dimensional anatomical volume based on the pose of the probe and based on the 3-dimensional imaging information; and
store the location in memory based on detecting the trigger and detecting the pose of the probe.
